(19) United States
(12) Patent Application Publication
(10) Pub. No.: US 2010/0131044 A1
(43) Pub. Date: May 27, 2010

(54) STENT

(76) Inventor: Udayan Patel, San Jose, CA (US)

Correspondence Address: FAY SHARPE LLP, 1228 Euclid Avenue, 5th Floor, The Halle Building, Cleveland, OH, 44115, US

(21) Appl. No.: 12/373,380

(22) PCT Filed: Jul. 13, 2007
(86) PCT No.: PCT/US07/16056

§ 371 Date: Jul. 9, 2009

Related U.S. Application Data
(60) Provisional application No. 60/831,981, filed on Jul. 13, 2006.

(51) Int. Cl. A61F 2/82

(52) U.S. Cl. 623/1.16

(57) ABSTRACT

An expandable stent for use within a body passageway. The stent includes at least two struts and a connector securing together said two struts. At least one of said struts includes an elbow section and an undulating section. The apex of at least one strut can include at least one a dimple, divot and/or slot.

This application claims priority on PCT Application Serial No. PCT/US2007/016056 filed Jul. 13, 2007, which in turn claims priority on U.S. Patent Application Ser. No. 60/831,981 filed on Jul. 13, 2006, which are both incorporated herein.

The invention relates generally to medical devices, and particularly to a stent for use in treating a body passageway.

### BACKGROUND OF THE INVENTION

Medical treatment of various illnesses or diseases commonly includes the use of one or more medical devices. One type of medical device that is commonly used to repair various types of body passageways is an expandable stent. One purpose of a stent is to open a blocked or partially blocked body passageway. When a stent is used in a blood vessel, the stent is used to open the occluded vessel to achieve improved blood flow which is necessary to provide for the anatomical function of an organ. The procedure of opening a blocked or partially blocked body passageway commonly includes the use of one or more stents in combination with other medical devices such as, but not limited to, an introducer sheath, a guiding catheter, a guide wire, an angioplasty balloon, etc.

Various physical attributes of a stent can contribute directly to the success rate of the device. These physical attributes include radiopacity, hoop strength, radial force, thickness of the metal, dimensions of the metal and the like. Cobalt and chromium and stainless steel are commonly used to form stents. These materials are commonly used since such materials having a known history of safety, effectiveness and biocompatibility. These materials however have limited physical performance characteristics as to size, strength, weight, bendability, biostability and radiopacity. As a result, new materials better properties than conventional materials such as stainless steel or cobalt alloys, but with lower ductility, have been developed.

The present invention is generally directed to a medical device such as, but not limited to, a stent, and more particularly to a stent that is at least partially formed of materials having a lower ductility than conventional materials.

### SUMMARY OF THE INVENTION

The present invention is directed to a medical device that can be formed from conventional materials or include new materials having a lower ductility than conventional materials such as stainless steel or cobalt alloys. The medical device is generally in the form of a stent for use in a body passageway. As used herein, the term “body passageway” is defined to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, subarachnoid space, and central and peripheral nerve conduits, etc.). The techniques employed to deliver the device to a treatment area include, but are not limited to angioplasty, vascular anastomoses, transplantation, implantation, surgical implantation, subcutaneous introduction, minimally invasive surgical procedures, interventional procedures, and any combinations thereof. For vascular applications, the term “body passageway” primarily refers to blood vessels and chambers in the heart. The device can be an expandable stent and/or graft suitable for endovascular delivery and expandable by a balloon and/or other means (e.g., by its own internal forces “self expandable”). The stent, graft, and/or other suitable device can have many shapes and forms. During expansion of a stent in a body passageway, most of the deformation of the stent occurs at the hinge point where much, if not all, of the stresses are concentrated. The stent design in accordance with the present invention allows for deformation to occur at the hinge points as well as along the length of the strut of the stent, thus reducing the maximum stress at the hinge point and distributing the stresses beyond the hinge point. The stent design also can make the stent more flexible. In one non-limiting embodiment of the invention, reduction of the maximum stress at the hinge point and distributing of the stresses beyond the hinge point can be in part accomplished by providing an undulating pattern along at least a portion of the length of one or more struts on the stent. The undulating pattern along the strut length makes the strut ring flexible. The enhanced flexibility of the strut thus achieved reduces the need for long articulations between the strut rings. This in turn allows more rings to be placed within a given length of the stent. Hence the open areas are reduced and the radial force is improved. The curved section of the undulation along the strut can expand due to the forces exerted during the opening of the stent. The expansion of the curved regions can reduce the strain at the hinges between the struts. This will allow materials with low ductility to be formed into a balloon expandable stent. The length of the straight segment of the undulating pattern at least partially determines the flexibility of the struts along the longitudinal axis of the strut. The longer the straight segments, the greater is the flexibility of the strut and strut ring. In another and/or additional non-limiting embodiment of the invention, reduction of the maximum stress at the hinge point and distributing of the stresses beyond the hinge point can be in part accomplished by reducing the strut width along the length of the strut thus causing it to bend at the narrowest region. In still another and/or additional non-limiting embodiment of the invention, reduction of the maximum stress at the hinge point and distributing of the stresses beyond the hinge point can be in part accomplished by reducing the connector width along the length of the connector thus causing it to bend at the narrowest region. One or more connectors are used to connect together two or more struts on the stent. In yet another and/or additional non-limiting embodiment of the invention, reduction of the maximum stress at the hinge point and distributing of the stresses beyond the hinge point can be in part accomplished by providing an undulating pattern along at least a portion of the length of one or more connectors.

In another and/or additional aspect of the present invention, the length of the connectors on the stent can be shortened without reducing stent flexibility. By being able to reduce to the connector length without adversely affecting the flexibility of the stent, the stent can be designed to accommodate a larger number of rings formed by the struts per unit length of the stent, thus reducing the open spaces in the body of the stent and also or alternatively increasing the radial strength of the stent.

In still another and/or additional aspect of the present invention, the one or more undulations on the stent can be designed to at least partially elongate during the expansion of the stent, thus reducing foreshortening of the stent.

In yet another and/or additional aspect of the present invention, each ring on the stent is formed by the connection of two of more struts. One or more connector are used to secure together two or more adjacently positioned rings of struts. A elbow or hinge section is positioned between two or more struts to connect the struts together. The elbow or hinge portion can be considered a part of the strut or considered to be separate from the strut. One or more undulating patterns are placed along the length of each strut. In one non-limiting embodiment of the invention, the undulation can be composed of straight segments connected by curved segments. In such a design, at least one straight segment and at least two curved segments are used to form an undulating segment along the length of the strut. The region of the strut that does not include the undulating portion can be straight or curved. In another and/or additional non-limiting embodiment of the invention, the width of the undulating segment can be less wide than that of the strut; however, this is not required. In one non-limiting aspect of this embodiment, the curved segments of the undulating pattern can be narrower that the straight segment. The curved segment of the undulation can be the narrowest at the apex or can be narrowest on the two sides of the apex; however, this is not required. In another non-limiting aspect of this embodiment, the curved segments of the undulating pattern can be wider than the straight segment. In still another and/or additional non-limiting aspect of this embodiment, the elbow or hinge portion on or between two struts can be narrowed by placing a divot at the outside edge of the apex. The elbow or hinge width would thus progressively increase around the curvature on both sides of the divot. This configuration would progressively dissipate stresses radiating from apex and around the hinge segment. As can be appreciated, a divot can be also or alternatively place applied to the curved section of the undulating pattern. In yet another and/or additional non-limiting aspect of this embodiment, the straight segment of the undulating pattern can be placed at an angle with respect to the general axis of the strut within which it is placed. In one non-limiting design, the straight segment of the undulating pattern can be placed perpendicular to the strut axis. One non-limiting purpose of the undulation in the strut is to provide an area to which some of the stresses at the hinge point can be diverted and thus reduce the overall strain at the hinge point. Also or alternatively, some of the stresses at the hinge point can be diverted to reduce the overall strain at the hinge point by reducing the width of the strut anywhere along its length at one or multiple points along the length of the strut (e.g., the mid-point of the strut, etc.).

In still yet another and/or additional aspect of the present invention, the stent is designed such that during the expansion of the stent, the elbow or hinge section of or on the strut expands as a pair of adjacent struts are pushed away from each other. The divots in the apex of the elbow hinge provides a space to dissipate the compressive forces along the outer edge of the elbow or hinge section. This space thus reduces the tensile elongation on the inside edge of the strut. The reduced expansion at the elbow or hinge section is compensated by the expansion of the undulating segment along the length of the strut. In particular, the curved segment of the undulating pattern is designed to expand. This expansion also results in increasing the length of the strut. The increase in strut length at least partially compensates for the reduction in height of the ring during expansion, thus reducing the foreshortening of the stent. Depending on the length of the straight segment and the width of the curved segment of the undulating section on the strut, as well as the placement of the undulating section along the length of the strut, the flexibility of the ring as well as the overall stent can be increased. This in turn provides versatility in reducing the length of the articulation between the strut rings and uniformly distributes the metal over the expanded stent. The new stent design enables the strut to be formed from high strength metal alloys with 1) increased radiopacity, 2) enhanced corrosion resistance, and/or 3) low ductility.

In another and/or additional aspect of the present invention, the angle of the straight segment of the undulating pattern can be obtuse or acute with respect to the strut. The selection of the angle of the straight segment will affect extent to which the undulation can be opened during stent expansion and also dictate the flexibility of the unexpanded stent.

In still another and/or additional aspect of the present invention, the undulation can include one or more straight segments alternating with curved segments.

In yet another and/or additional aspect of the present invention, the undulations can be placed either at the center of the strut or anywhere along the length of the strut.

In still yet another and/or additional aspect of the present invention, the undulating pattern can be replaced by simply narrowing of the struts, but such substitution can reduce the benefit of compensating for foreshortening or increasing flexibility of stent. The narrowing can be in or more places.

In another and/or additional aspect of the present invention, the hinge section can be made from a more ductile material and the struts can be made from high strength, less ductile material. Such an arrangement can reduce or eliminate the need to distribute the strain at the hinge region and/or can reduce or eliminate the need for undulating segment. As can be appreciated, the stent can be formed of a uniform material.

In another and/or additional aspect of the present invention, the stent is designed such that the elbow or hinge section of or on the strut includes one or more divots and/or dimples. The one or more divots and/or dimples in the elbow or hinge section of or on the strut is designed to improve stress distribution over the elbow or hinge section of or on the strut during expansion and/or crimping of the stent. The one or more dimples in the elbow or hinge section of or on the strut form a thicker section in the elbow or hinge section of or on the strut such that when the elbow or hinge section of or on the strut is expanded and/or crimped the stresses on the elbow or hinge section of or on the strut are distributed on both sides of the dimple rather than in a single location on the elbow or hinge section of or on the strut. This distribution of stresses thus prevent the concentration of stress in a single location on the elbow or hinge section of or on the strut. Without the dimple on the elbow or hinge section of or on the strut, the stresses on the elbow or hinge section of or on the strut during expansion and/or crimping generally occur at or near the apex of the elbow or hinge section of or on the strut. By causing the stresses to be distributed on two of more regions on the elbow or hinge section of or on the strut, the occurrence of one or more portions of the elbow or hinge section of or on the strut exceeding the maximum strain limit of the material during expansion and/or crimping of the elbow or hinge section of or on the strut is significantly reduced or prevented. As such, thinner materials can be used for the stent, if so desired, when the dimple concept of the present invention is employed. In one non-limiting embodiment, the elbow or hinge section of or on the strut includes a single dimple at or closely adjacent to the apex of the elbow or hinge section of or on the strut. Typically the dimple is positioned on the back side of the apex of the elbow or hinge section of or on the strut to provide better stress distribution during the expansion of the elbow or hinge section of or on the strut; however, the dimple can be positioned on the front side of the apex. The thickness of the dimple is generally about 1-80% of the thickness of the elbow or hinge section of or on the strut that does not include the dimple, and about 4-50% of the thickness of the elbow or hinge section of or on the strut that does not include the dimple, and more typically about 10-40% of the thickness of the elbow or hinge section of or on the strut that does not include the dimple. The width of the dimple is generally greater than the thickness of the dimple. Generally, the ratio of the width to thickness of the dimple is about 1.01-10:1, typically about 1.05-5:1. In another and/or additional non-limiting embodiment, the elbow or hinge section of or on the strut includes a plurality of dimples at or closely adjacent to the apex of the elbow or hinge section of or on the strut. Typically the dimples are all positioned on the back side of the apex of the elbow or hinge section of or on the strut to provide better stress distribution during the expansion of the elbow or hinge section of or on the strut; however, one or more or all of the dimples can be positioned on the front side of the apex. The thickness of the dimples is generally about 1-80% of the thickness of the elbow or hinge section of or on the strut that does not include the dimple, and about 4-50% of the thickness of the elbow or hinge section of or on the strut that does not include the dimple, and more typically about 10-40% of the thickness of the elbow or hinge section of or on the strut that does not include the dimple. The thickness of the dimples can be the same or different. The width of the dimples is generally greater than the thickness of the dimple. Generally, the ratio of the width to thickness of the dimple is about 1.01-10:1, typically about 1.05-5:1. The width of the dimples can be the same or different. In still another and/or alternative non-limiting embodiment, the elbow or hinge section of or on the strut includes a single divot at or closely adjacent to the apex of the elbow or hinge section of or on the strut. Typically the divot is positioned on the front side of the apex of the elbow or hinge section of or on the strut to provide better stress distribution during the expansion of the elbow or hinge section of or on the strut; however, the divot can be positioned on the back side of the apex. When the elbow or hinge section of or on the strut includes a single dimple, the single divot is generally positioned on the opposite side of the dimple and positioned directly across from the dimple; however, this is not required. The depth of the divot is generally about 1-80% of the thickness of the elbow or hinge section of or on the strut that does not include the divot, and about 4-50% of the thickness of the elbow or hinge section of or on the strut that does not include the divot, and more typically about 10-40% of the thickness of the elbow or hinge section of or on the strut that does not include the divot. The width of the divot is generally greater than the depth of the divot. Generally, the ratio of the width to depth of the divot is about 1.01-10:1, typically about 1.05-5:1. In yet another and/or additional non-limiting embodiment, the elbow or hinge section of or on the strut includes a plurality of divots at or closely adjacent to the apex of the elbow or hinge section of or on the strut. Typically the divots are all positioned on the front side of the apex of the elbow or hinge section of or on the strut to provide better stress distribution during the expansion of the elbow or hinge section of or on the strut; however, one or more or all of the divots can be positioned on the back side of the apex. The depth of the divots is generally about 1-80% of the thickness of the elbow or hinge section of or on the strut that does not include the divot, and about 4-50% of the thickness of the elbow or hinge section of or on the strut that does not include the divot, and more typically about 10-40% of the thickness of the elbow or hinge section of or on the strut that does not include the divot. The depth of the divots can be the same or different. The width of the divots is generally greater than the depth of the divot. Generally, the ratio of the width to depth of the divots is about 1.01-10:1, typically about 1.05-5:1. The width of the divots can be the same or different. When two divots are included on the elbow or hinge section of or on the strut, a dimple is generally positioned at least partially between the divots and on the opposite side of the elbow or hinge section of or on the strut from the divots; however, this is not required.

In still another and/or additional aspect of the present invention, the stent is designed such that the elbow or hinge section of or on the strut includes one or more slits. The one or more slits are designed to a) facilitate in the crimping of the stent, and/or b) facilitate in the expansion of the stent. The one or more slits create one or more narrow points in the elbow or hinge section of or on the strut to increase the ease of crimping the stent and/or to reduces stresses on the elbow or hinge section of or on the strut during the crimping and/or expansion of the stent. During the expansion of the stent, the one or more slits increase the flexibility of the elbow or hinge section of or on the strut to some point of expansion and thereafter the sides of the slit contact one another, thereby reducing the further flexibility of the elbow or hinge section of or on the strut. Generally the one or more slits are located on the front surface of the elbow or hinge section of or on the strut; however, this is not required. In addition, the one or more slits are generally located at or near the apex of the elbow or hinge section of or on the strut; however, it can be appreciated that one or more slits can be positioned on other or additional regions on the strut and/or the elbow or hinge section of or on the strut. A dimple can be positioned on the opposite side of the slit on the strut and/or the elbow or hinge section of or on the strut; however, this is not required. The use of the dimple in combination with the slit results in desired flexibility from the slit and desired stress distribution from the dimple. The depth of the one or more slits is generally about 1-80% of the thickness of the elbow or hinge section of or on the strut that does not include the slit, and about 4-50% of the thickness of the elbow or hinge section of or on the strut that does not include the slit, and more typically about 10-40% of the thickness of the elbow or hinge section of or on the strut that does not include the slit. The depth of the two of more slits can be the same or different. The width of the slits is generally less than the depth of the divot. Generally, the ratio of the depth to the width of the slits is about 1.01-10:1, typically about 1.05-5:1. The width of two or more slits can be the same or different.

In still another and/or additional aspect of the present invention, the stent is at least partially made of a metal alloy that improves one or more properties (e.g., strength, durability, hardness, biostability, bendability, coefficient of friction, radial strength, flexibility, tensile strength, tensile elongation, longitudinal lengthening, stress-strain properties, improved recoil properties, radiopacity, heat sensitivity, biocompatibility, etc.) of the stent. The metal alloy that is used to at least partially form the stent can 1) increase the radiopacity of the stent, 2) increase the radial strength of the stent, 3) increase the yield strength and/or ultimate tensile strength of the stent, 4) improve the stress-strain properties of the stent, 5) improve the crimping and/or expansion properties of the stent, 6) improve the bendability and/or flexibility of the stent, 7) improve the strength and/or durability of the stent, 8) increase the hardness of the stent, 9) improve the longitudinal lengthening properties of the stent, 10) improved the recoil properties of the stent, 11) improve the friction coefficient of the stent, 12) improve the heat sensitivity properties of the stent, 13) improve the biostability and/or biocompatibility properties of the stent, and/or 14) enable smaller, thinner and/or lighter weight stent to be made. The stent can be formed by one or more manufacturing processes such as, but are not limited to, laser cutting, electrical discharge machining (EDM), etching, crimping, annealing, drawing, pilgering, electroplating, electro-polishing, chemical polishing, cleaning, pickling, ion beam deposition or implantation, sputter coating, vacuum deposition, wire welding, etc.

In yet another and/or alternative non-limiting aspect of the present invention, the novel metal alloy that is used to form all or a portion of the stent includes rhenium and molybdenum. The novel alloy can include one or more other metals such as, but not limited to, calcium, chromium, cobalt, copper, gold, iron, lead, magnesium, nickel, niobium, platinum, rare earth metals, silver, tantalum, titanium, tungsten, yttrium, zinc, zirconium, and/or alloys thereof.

In still yet another and/or alternative non-limiting aspect of the present invention, the stent can include, contain and/or be coated with one or more chemical agents that facilitate in the success of the stent and/or treated area. The stent can include, contain and/or be coated with one or more chemical agents that inhibit or prevent in-stent restenosis, vascular narrowing, and/or thrombosis during and/or after the stent is inserted into a treatment area; however, this is not required. In addition or alternatively, the stent can include, contain and/or be coated with one or more chemical agents that can be used in conjunction with the one or more chemical agents that inhibit or prevent in-stent restenosis, vascular narrowing, and/or thrombosis that are included in, contained in and/or coated in the stent. As such, the stent, when it includes, contains, and/or is coated with one or more chemical agents, can include one or more chemical agents to address one or more medical needs. As such, the stent can include, contain and/or be coated with one or more chemical agents that include, but are not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to viral, fungus and/or bacteria infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. In one non-limiting embodiment, the one or more chemical agents that can be include with, contained in and/or be coated on the stent include, but are not limited to, an anti-platelet compound and/or anticoagulant compound such as, but not limited to, warfarin (Coumadin), warfarin derivatives, aspirin, aspirin derivatives, clopidogrel, clopidogrel derivatives, ticlopadine, ticlopadine derivatives, hirdun, hirdun derivatives, dipyridamole, dipyridamole derivatives, trapidil, trapidil derivatives, taxol, taxol derivatives, cytochalasin, cytochalasin derivatives, paclitaxel, paclitaxel derivatives, rapamycin, rapamycin derivatives, 5-Phenylmethimazole, 5-Phenylmethimazole derivatives, GM-CSF, GM-CSF derivatives, heparin, heparin derivatives, low molecular weight heparin, low molecular weight heparin derivatives, or combinations thereof. One specific non-limiting example of an anti-thrombotic inhibitor that can be include with, contained in and/or be coated on the stent includes 1) huridin and/or derivatives, and/or 2) alagors (e.g., bivalirudin, etc.) and/or derivatives. As can be appreciated, one or more other anti-thrombotic chemical agents can be used with the stent. Non-limiting examples of chemical agents that can be used include, but are not limited to, 5-Fluorouracil and/or derivatives thereof; 5-Phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; chemical agents that modulate intracellular Ca2+ transport such as L-type (e.g., diltiazem, nifedipine, verapamil, etc.) or T-type Ca2+ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; anti-biotics; anti-coagulant compounds and/or derivatives thereof; anti-fibrosis compounds and/or derivatives thereof; antifungal compounds and/or derivatives thereof; anti-inflammatory compounds and/or derivatives thereof; Anti-Invasive Factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative and/or derivatives thereof; anti-thrombogenic agents and/or derivatives thereof; argatroban and/or derivatives thereof; ap-1 inhibitors and/or derivatives thereof (e.g., for tyrosine kinase, protein kinase C, myosin light chain kinase, Ca2+/calmodulin kinase II, casein kinase II, etc.); aspirin and/or derivatives thereof; azathioprine and/or derivatives thereof; β-Estradiol and/or derivatives thereof; β-1-anticollagenase and/or derivatives thereof; calcium channel blockers and/or derivatives thereof; calmodulin antagonists and/or derivatives thereof (e.g., H7, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP3 receptor and/or derivatives thereof; inhibitors of the sodium/hydrogen antiporter and/or derivatives thereof (e.g., amiloride and derivatives thereof, etc.); insulin and/or derivatives thereof; Interferon alpha 2 Macroglobulin and/or derivatives thereof; ketoconazole and/or derivatives thereof; Lepirudin and/or derivatives thereof; LISINOPRIL and/or derivatives thereof; LOVASTATIN and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; Plasminogen Activator Inhibitor-1 and/or derivatives thereof; Plasminogen Activator Inhibitor-2 and/or derivatives thereof; Platelet Factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H3P32O4, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof; TH1 and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; Tissue Inhibitor of Metalloproteinase-1 and/or derivatives thereof; Tissue Inhibitor of Metalloproteinase-2 and/or derivatives thereof; tissue plasma activators; TNF and/or derivatives thereof; tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof. As can be appreciated, the chemical agent can include one or more derivatives of the above listed compounds and/or other compounds. In one non-limiting embodiment, the chemical agent includes, but is not limited to, trapidil, Trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, 5-Phenylmethimazole, 5-Phenylmethimazole derivatives, GM-CSF (granulo-cytemacrophage colony-stimulating-factor), GM-CSF derivatives, statins or HMG-CoA reductase inhibitors forming a class of hypolipidemic agents, combinations, or analogs thereof, or combinations thereof. The type and/or amount of chemical agent included in the device and/or coated on the device can vary. When two or more chemical agents are included in and/or coated on the device, the amount of two or more chemical agents can be the same or different. The type and/or amount of chemical agent included on, in and/or in conjunction with the device are generally selected to address one or more clinical events. Typically the amount of chemical agent included on, in and/or used in conjunction with the device is about 0.01-100 ug per mm² and/or at least about 0.01 weight percent of device; however, other amounts can be used. In one non-limiting embodiment of the invention, the device can be partially of fully coated and/or impregnated with one or more chemical agents to facilitate in the success of a particular medical procedure. The amount of two of more chemical agents on, in and/or used in conjunction with the device can be the same or different. The one or more chemical agents can be coated on and/or impregnated in the device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the invention, the type and/or amount of chemical agent included on, in and/or in conjunction with the device is generally selected for the treatment of one or more clinical events. Typically the amount of chemical agent included on, in and/or used in conjunction with the device is about 0.01-100 ug per mm² and/or at least about 0.01-100 weight percent of the device; however, other amounts can be used. The amount of two of more chemical agents on, in and/or used in conjunction with the device can be the same or different. For instance, portions of the device to provide local and/or systemic delivery of one or more chemical agents in and/or to a body passageway to a) inhibit or prevent thrombosis, in-stent restenosis, vascular narrowing and/or restenosis after the device has been inserted in and/or connected to a body passageway, b) at least partially passivate, remove, encapsulate, and/or dissolve lipids, fibroblast, fibrin, etc. in a body passageway so as to at least partially remove such materials and/or to passivate such vulnerable materials (e.g., vulnerable plaque, etc.) in the body passageway in the region of the device and/or downstream of the device. As can be appreciated, the one or more chemical agents can have many other or additional uses. In still another and/or alternative non-limiting example, the device is coated with and/or includes one or more chemical agents such as, but not limited to chemical agents associated with thrombolytics, vasodilators, anti-hypertensive agents, antimicrobial or anti-biotic, anti-mitotic, anti-proliferative, anti-secretory agents, non-steroidal anti-inflammatory drugs, immunosuppressive agents, growth factors and growth factor antagonists, endothelial growth factors and growth factor antagonists, antitumor and/or chemotherapeutic agents, anti-polymerases, anti-viral agents, anti-body targeted therapy agents, hormones, anti-oxidants, biologic components, radio-therapeutic agents, radiopaque agents and/or radio-labeled agents. In addition to these chemical agents, the device can be coated with and/or include one or more chemical agents that are capable of inhibiting or preventing any adverse biological response by and/or to the device that could possibly lead to device failure and/or an adverse reaction by human or animal tissue. A wide range of chemical agents thus can be used. The one or more chemical agents can be coated on and/or impregnated in the stent by a variety of mechanisms such as, but not limited to, spraying (e.g., atomizing spray techniques, etc.), dip coating, roll coating, sonication, brushing, plasma deposition, depositing by vapor deposition.

In a further and/or alternative non-limiting aspect of the present invention, the one or more chemical agents on and/or in the stent, when used on the stent, can be released in a controlled manner so the area in question to be treated is provided with the desired dosage of chemical agent over a sustained period of time. As can be appreciated, controlled release of one or more chemical agents on the stent is not always required and/or desirable. As such, one or more of the chemical agents on and/or in the stent can be uncontrollably released from the stent during and/or after insertion of the stent in the treatment area. It can also be appreciated that one or more chemical agents on and/or in the stent can be controllably released from the stent and one or more chemical agents on and/or in the stent can be uncontrollably released from the stent. It can also be appreciated that one or more chemical agents on and/or in one region of the stent can be controllably released from the stent and one or more chemical agents on and/or in the stent can be uncontrollably released from another region on the stent. As such, the stent can be designed such that 1) all the chemical agent on and/or in the stent is controllably released, 2) some of the chemical agent on and/or in the stent is controllably released and some of the chemical agent on the stent is non-controllably released, or 3) none of the chemical agent on and/or in the stent is controllably released. The stent can also be designed such that the rate of release of the one or more chemical agents from the stent is the same or different. The stent can also be designed such that the rate of release of the one or more chemical agents from one or more regions on the stent is the same or different. Non-limiting arrangements that can be used to control the release of one or more chemical agent from the stent include a) at least partially coat one or more chemical agents with one or more polymers, b) at least partially incorporate and/or at least partially encapsulate one or more chemical agents into and/or with one or more polymers, and/or c) insert one or more chemical agents in pores, passageway, cavities, etc. in the stent and at least partially coat or cover such pores, passageway, cavities, etc. with one or more polymers. As can be appreciated, other or additional arrangements can be used to control the release of one or more chemical agent from the stent. The one or more polymers used to at least partially control the release of one or more chemical agent from the stent can be porous or non-porous. The one or more chemical agents can be inserted into and/or applied to one or more surface structures and/or micro-structures on the stent, and/or be used to at least partially form one or more surface structures and/or micro-structures on the stent. As such, the one or more chemical agents on the stent can be 1) coated on one or more surface regions of the stent, 2) inserted and/or impregnated in one or more surface structures and/or micro-structures, etc. of the stent, and/or 3) form at least a portion or be included in at least a portion of the structure of the stent. When the one or more chemical agents are coated on the stent, the one or more chemical agents can 1) be directly coated on one or more surfaces of the stent, 2) be mixed with one or more coating polymers or other coating materials and then at least partially coated on one or more surfaces of the stent, 3) be at least partially coated on the surface of another coating material that has been at least partially coated on the stent, and/or 4) be at least partially encapsulated between a) a surface or region of the stent and one or more other coating materials and/or b) two or more other coating materials. As can be appreciated, many other coating arrangements can be additionally or alternatively used. When the one or more chemical agents are inserted and/or impregnated in one or more internal structures, surface structures and/or micro-structures of the stent, 1) one or more other coating materials can be applied at least partially over the one or more internal structures, surface structures and/or micro-structures of the stent, and/or 2) one or more polymers can be combined with one or more chemical agents. As such, the one or more chemical agents can be 1) embedded in the structure of the stent; 2) positioned in one or more internal structures of the stent; 3) encapsulated between two polymer coatings; 4) encapsulated between the base structure and a polymer coating; 5) mixed in the base structure of the stent that includes at least one polymer coating; or 6) one or more combinations of 1, 2, 3, 4 and/or 5. In addition or alternatively, the one or more coating of the one or more polymers on the stent can include 1) one or more coatings of non-porous polymers; 2) one or more coatings of a combination of one or more porous polymers and one or more non-porous polymers; 3) one or more coating of porous polymer, or 4) one or more combinations of options 1, 2, and 3. As can be appreciated different chemical agents can be located in and/or between different polymer coating layers and/or on and/or the structure of the stent. As can also be appreciated, many other and/or additional coating combinations and/or configurations can be used. The concentration of one or more chemical agents, the type of polymer, the type and/or shape of internal structures in the stent and/or the coating thickness of one or more chemical agents can be used to control the release time, the release rate and/or the dosage amount of one or more chemical agents; however, other or additional combinations can be used. As such, the chemical agent and polymer system combination and location on the stent can be numerous. As can also be appreciated, one or more chemical agents can be deposited on the top surface of the stent to provide an initial uncontrolled burst effect of the one or more chemical agents prior to 1) the control release of the one or more chemical agents through one or more layers of polymer system that include one or more non-porous polymers and/or 2) the uncontrolled release of the one or more chemical agents through one or more layers of polymer system. The one or more chemical agents and/or polymers can be coated on the stent by a variety of mechanisms such as, but not limited to, spraying (e.g., atomizing spray techniques, etc.), dip coating, roll coating, sonication, brushing, plasma deposition, and/or depositing by vapor deposition. The thickness of each polymer layer and/or layer of chemical agent is generally at least about 0.01 μm and is generally less than about 150 μm. The one or more chemical agents on and/or in the device, when used on the device, can be released in a controlled manner so the area in question to be treated is provided with the desired dosage of chemical agent over a sustained period of time. As can be appreciated, controlled release of one or more chemical agents on the device is not always required and/or desirable. As such, one or more of the chemical agents on and/or in the device can be uncontrollably released from the device during and/or after insertion of the device in the treatment area. It can also be appreciated that one or more chemical agents on and/or in the device can be controllably released from the device and one or more chemical agents on and/or in the device can be uncontrollably released from the device. It can also be appreciated that one or more chemical agents on and/or in one region of the device can be controllably released from the device and one or more chemical agents on and/or in the device can be uncontrollably released from another region on the device. As such, the device can be designed such that 1) all the chemical agent on and/or in the device is controllably released, 2) some of the chemical agent on and/or in the device is controllably released and some of the chemical agent on the device is non-controllably released, or 3) none of the chemical agent on and/or in the device is controllably released. The device can also be designed such that the rate of release of the one or more chemical agents from the device is the same or different. The device can also be designed such that the rate of release of the one or more chemical agents from one or more regions on the device is the same or different. Non-limiting arrangements that can be used to control the release of one or more chemical agent from the device include a) at least partially coat one or more chemical agents with one or more polymers, b) at least partially incorporate and/or at least partially encapsulate one or more chemical agents into and/or with one or more polymers, c) insert one or more chemical agents in pores, passageway, cavities, etc. in the device and at least partially coat or cover such pores, passageway, cavities, etc. with one or more polymers, and/or incorporate one or more chemical agents in the one or more polymers that at least partially form the device. As can be appreciated, other or additional arrangements can be used to control the release of one or more chemical agent from the device. The one or more polymers used to at least partially control the release of one or more chemical agent from the device can be porous or non-porous. The one or more chemical agents can be inserted into and/or applied to one or more surface structures and/or micro-structures on the device, and/or be used to at least partially form one or more surface structures and/or micro-structures on the device. As such, the one or more chemical agents on the device can be 1) coated on one or more surface regions of the device, 2) inserted and/or impregnated in one or more surface structures and/or micro-structures, etc. of the device, and/or 3) form at least a portion or be included in at least a portion of the structure of the device. When the one or more chemical agents are coated on the device, the one or more chemical agents can, but is not required to, 1) be directly coated on one or more surfaces of the device, 2) be mixed with one or more coating polymers or other coating materials and then at least partially coated on one or more surfaces of the device, 3) be at least partially coated on the surface of another coating material that has been at least partially coated on the device, and/or 4) be at least partially encapsulated between a) a surface or region of the device and one or more other coating materials and/or b) two or more other coating materials. As can be appreciated, many other coating arrangements can be additionally or alternatively used. When the one or more chemical agents are inserted and/or impregnated in one or more portions of the device, one or more surface structure and/or micro-structures of the device, and/or one or more surface structures and/or micro-structures of the device, 1) one or more other polymers can be applied at least partially over the one or more surface structure and/or micro-structures, surface structures and/or micro-structures of the device, 2) one or more polymers can be combined with one or more chemical agents, and/or 3) one or more polymers can be coated over or more portions of the body of the device; however, this is not required. As such, the one or more chemical agents can be 1) embedded in the structure of the device; 2) positioned in one or more surface structure and/or micro-structures of the device; 3) encapsulated between two polymer coatings; 4) encapsulated between the base structure and a polymer coating; 5) mixed in the base structure of the device that includes at least one polymer coating; or 6) one or more combinations of 1, 2, 3, 4 and/or 5. In addition or alternatively, the one or more coatings of the one or more polymers on the device can include 1) one or more coatings of non-porous polymers; 2) one or more coatings of a combination of one or more porous polymers and one or more non-porous polymers; 3) one or more coating of porous polymer, or 4) one or more combinations of options 1, 2, and 3. As can be appreciated different chemical agents can be located in and/or between different polymer coating layers and/or on and/or the structure of the device. As can also be appreciated, many other and/or additional coating combinations and/or configurations can be used. In a further and/or alternative non-limiting embodiment of the present invention, the device can be embedded with and/or impregnated with one or more chemical agents using a solvent to temporarily and/or permanently increase the porosity of the structure of a non-porous and/or porous polymer coating and/or device and be used to transport one or more chemical agents into the matrix of the device. One or more solvents can be used to transport one or more chemical agents. Solvent suitability is a function of compatibility with one or more chemical agents and one or more materials of the device. Non-limiting examples of solvents include Dimethyl sulfoxide (DMSO), chloroform, ethylene, methanol, ethyl acetate, and the broader class of biocompatible or non-biocompatible solvents. The concentration of one or more chemical agents, the type of polymer, the type and/or shape of surface structure and/or micro-structures in the device and/or the coating thickness of one or more chemical agents can be used to control the release time, the release rate and/or the dosage amount of one or more chemical agents; however, other or additional combinations can be used. As such, the chemical agent and polymer system combination and location on the device can be numerous. As can also be appreciated, one or more chemical agents can be deposited on the top surface of the device to provide an initial uncontrolled burst effect of the one or more chemical agents prior to 1) the control release of the one or more chemical agents through one or more layers of polymer system that include one or more nonporous polymers and/or 2) the uncontrolled release of the one or more chemical agents through one or more layers of polymer system. The one or more chemical agents and/or polymers can be coated on and/or impregnated in the device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. The thickness of each polymer layer and/or layer of chemical agent is generally at least about 0.01 μm and is generally less than about 150 μm. In one non-limiting embodiment, the thickness of a polymer layer and/or layer of chemical agent is about 0.02-75 μm, more particularly about 0.05-50 μm, and even more particularly about 1-30 μm. When the device includes and/or is coated with one or more chemical agents such that at least one of the chemical agents is at least partially controllably released from the device, the need or use of body-wide therapy for extended periods of time can be reduced or eliminated. In the past, the use of body-wide therapy was used by the patient long after the patient left the hospital or other type of medical facility. This body-wide therapy could last days, weeks, months or sometimes over a year after surgery. The device of the present invention can be applied or inserted into a treatment area and 1) merely requires reduced use and/or extended use of systemic therapy after application or insertion of the device or 2) does not require use and/or extended use of systemic therapy after application or insertion of the device. As can be appreciated, use and/or extended use of systemic therapy can be used after application or insertion of the device at the treatment area. In one non-limiting example, no body-wide therapy is needed after the insertion of the device into a patient. In another and/or alternative non-limiting example, short term use of systemic therapy is needed or used after the insertion of the device into a patient. Such short term use can be terminated after the release of the patient from the hospital or other type of medical facility, or one to two days or weeks after the release of the patient from the hospital or other type of medical facility; however, it will be appreciated that other time periods of systemic therapy can be used. As a result of the use of the device of the present invention, the use of systemic therapy after a medical procedure involving the insertion of a device into a treatment area can be significantly reduced or eliminated.

In another and/or alternative non-limiting aspect of the present invention, controlled release of one or more chemical agents from the device, when controlled release is desired, can be accomplished by using one or more non-porous polymer layers and/or by use of one or more biodegradable polymers used to at least partially form the device; however, other and/or additional mechanisms can be used to controllably release the one or more chemical agents. The one or more chemical agents can be at least partially controllably released by molecular diffusion through the one or more non-porous polymer layers and/or from the one or more biodegradable polymers used to at least partially form the device. When one or more non-porous polymer layers are used, the one or more polymer layers are typically biocompatible polymers; however, this is not required. One or more non-porous polymers can be applied to the device without the use of chemical, solvents, and/or catalysts; however, this is not required. In one non-limiting example, the non-porous polymer can be at least partially applied by, but not limited to, vapor deposition and/or plasma deposition. The non-porous polymer can be selected so as to polymerize and cure merely upon condensation from the vapor phase; however, this is not required. The application of the one or more nonporous polymer layers can be accomplished without increasing the temperature above ambient temperature (e.g., 65-90° F.); however, this is not required. The non-porous polymer system can be mixed with one or more chemical agents prior to being formed into at least a portion of the device and/or be coated on the device, and/or be coated on a device that previously included one or more chemical agents; however, this is not required. The use or one or more non-porous polymers allows for accurate controlled release of the chemical agent from the device. The controlled release of one or more chemical agents through the nonporous polymer is at least partially controlled on a molecular level utilizing the motility of diffusion of the chemical agent through the non-porous polymer. In one non-limiting example, the one or more non-porous polymer layers can include, but are not limited to, polyamide, parylene (e.g., parylene C, parylene N) and/or a parylene derivative.

In still another and/or alternative non-limiting aspect of the present invention, controlled release of one or more chemical agents from the device, when controlled release is desired, can be accomplished by using one or more polymers that form a chemical bond with one or more chemical agents. In one non-limiting example, at least one chemical agent includes trapidil, trapidil derivative or a salt thereof that is covalently bonded to at least one polymer such as, but not limited to, an ethylene-acrylic acid copolymer. The ethylene is the hydrophobic group and acrylic acid is the hydrophilic group. The mole ratio of the ethylene to the acrylic acid in the copolymer can be used to control the hydrophobicity of the copolymer. The degree of hydrophobicity of one or more polymers can also be used to control the release rate of one or more chemical agents from the one or more polymers. The amount of chemical agent that can be loaded with one or more polymers may be a function of the concentration of anionic groups and/or cationic groups in the one or more polymers. For chemical agents that are anionic, the concentration of chemical agent that can be loaded on the one or more polymers is generally a function of the concentration of cationic groups (e.g. amine groups and the like) in the one or more polymer and the fraction of these cationic groups that can ionically bind to the anionic form of the one or more chemical agents. For chemical agents that are cationic (e.g., trapidil, etc.), the concentration of chemical agent that can be loaded on the one or more polymers is generally a function of the concentration of anionic groups (i.e., carboxylate groups, phosphate groups, sulfate groups, and/or other organic anionic groups) in the one or more polymers, and the fraction of these anionic groups that can ionically bind to the cationic form of the one or more chemical agents. As such, the concentration of one or more chemical agents that can be bound to the one or more polymers can be varied by controlling the amount of hydrophobic and hydrophilic monomer in the one or more polymers, by controlling the efficiency of salt formation between the chemical agent, and/or the anionic/cationic groups in the one or more polymers. In still another and/or alternative non-limiting aspect of the present invention, controlled release of one or more chemical agents from the device, when controlled release is desired, can be accomplished by using one or more polymers that include one or more induced cross-links. These one or more cross-links can be used to at least partially control the rate of release of the one or more chemical agents from the one or more polymers. The cross-linking in the one or more polymers can be instituted by a number of techniques such as, but not limited to, using catalysts, using radiation, using heat, and/or the like. The one or more cross-links formed in the one or more polymers can result in the one or more chemical agents to become partially or fully entrapped within the cross-linking, and/or form a bond with the cross-linking. As such, the partially or fully chemical agent takes longer to release itself from the crosslinking, thereby delaying the release rate of the one or more chemical agents from the one or more polymers. Consequently, the amount of chemical agent, and/or the rate at which the chemical agent is released from the device over time can be at least partially controlled by the amount or degree of cross-linking in the one or more polymers. In still a further and/or alternative aspect of the present invention, a variety of polymers can be coated on the device and/or be used to form at least a portion of the device. The one or more polymers can be used on the medical for a variety of reasons such as, but not limited to, 1) forming a portion of the device, 2) improving a physical property of the device (e.g., improve strength, improve durability, improve biocompatibility, reduce friction, etc.), 3) forming a protective coating on one or more surface structures on the device, 4) at least partially forming one or more surface structures on the stent, and/or 5) at least partially controlling a release rate of one or more chemical agents from the device. As can be appreciated, the one or more polymers can have other or additional uses on the device. The one or more polymers can be porous, non-porous, biostable, biodegradable (i.e., dissolves, degrades, is absorbed, or any combination thereof in the body), and/or biocompatible. When the device is coated with one or more polymers, the polymer can include 1) one or more coatings of non-porous polymers; 2) one or more coatings of a combination of one or more porous polymers and one or more non-porous polymers; 3) one or more coatings of one or more porous polymers and one or more coatings of one or more non-porous polymers; 4) one or more coatings of porous polymer, or 5) one or more combinations of options 1, 2, 3 and 4. The thickness of one or more of the polymer layers can be the same or different. When one or more layers of polymer are coated onto at least a portion of the device, the one or more coatings can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers that can be coated on the device and/or used to at least partially form the device can be polymers that are considered to be biodegradable; polymers that are considered to be biostable; and/or polymers that can be made to be biodegradable and/or biodegradable with modification. Non-limiting examples of polymers that are considered to be biodegradable include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g., poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-covalerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethylester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/copolymers, blends, and/or composites of above. Non-limiting examples of polymers that considered to be biostable include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ether ketone) (PEEK); poly(styrene-b-isobutylene-bstyrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g. polystyrene); poly(vinyl ethers) (e.g. polyvinyl methyl ether); poly(vinyl ketones); poly(vinylidene halides) (e.g. polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g. polyvinyl chloride); polyacrylic acid; ethylene acrylic acid copolymer; ethylene vinyl acetate copolymer; polyvinyl alcohol; poly(hydroxyl alkylmethacrylate); Polyvinyl esters (e.g. polyvinyl acetate); and/or copolymers, blends, and/or composites of above. Non-limiting examples of polymers that can be made to be biodegradable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used. The one or more polymers can be coated on and/or impregnated in the device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold. The thickness of each polymer layer is generally at least about 0.01 μm and is generally less than about 150 μm; however, other thicknesses can be used. In one non-limiting embodiment, the thickness of a polymer layer and/or layer of chemical agent is about 0.02-75 μm, more particularly about 0.05-50 μm, and even more particularly about 1-30 μm. As can be appreciated, other thicknesses can be used. In one non-limiting embodiment, that at least a portion of the body includes and/or is coated with parylene, PLGA, POE, PGA, PLLA, PAA, PEG, chitosan and/or copolymers, blends, and/or composites of above and/or derivatives of one or more of these polymers. In another and/or alternative non-limiting embodiment, at least a portion of the body includes and/or is coated with a nonporous polymer that includes, but is not limited to, polyamide, parylene c, parylene n and/or a parylene derivative. In still another and/or alternative non-limiting embodiment, at least a portion of the body includes and/or is coated with poly(ethylene oxide), poly(ethylene glycol), and poly(propylene oxide), polymers of silicone, methane, tetrafluoroethylene (including TEFLON brand polymers), tetramethyldisiloxane, and the like.

In another and/or alternative non-limiting aspect of the present invention, the stent, when including and/or is coated with one or more chemical agents, can include and/or can be coated with one or more chemical agents that are the same or different in different regions of the stent and/or have differing amounts and/or concentrations in differing regions of the stent. For instance, the stent can a) be coated with and/or include one or more biologicals on at least one portion of the stent and at least another portion of the stent is not coated with and/or includes biological agent; b) be coated with and/or include one or more biologicals on at least one portion of the stent that is different from one or more biologicals on at least another portion of the stent; c) be coated with and/or include one or more biologicals at a concentration on at least one portion of the stent that is different from the concentration of one or more biologicals on at least another portion of the stent; etc.

In still another and/or alternative non-limiting aspect of the present invention, one or more surfaces of the stent can be treated to achieve the desired coating properties of the one or more chemical agents and one or more polymers coated on the stent. Such surface treatment techniques include, but are not limited to, cleaning, buffing, smoothing, etching (chemical etching, plasma etching, etc.), etc. When an etching process is used, various gasses can be used for such a surface treatment process such as, but not limited to, carbon dioxide, nitrogen, oxygen, Freon, helium, hydrogen, etc. The plasma etching process can be used to clean the surface of the stent, change the surface properties of the stent so as to affect the adhesion properties, lubricity properties, etc. of the surface of the stent. As can be appreciated, other or additional surface treatment processes can be used prior to the coating of one or more chemical agents and/or polymers on the surface of the stent. In one non-limiting manufacturing process, one or more portions of the stent are cleaned and/or plasma etched; however, this is not required. Plasma etching can be used to clean the surface of the stent, and/or to form one or more non-smooth surfaces on the stent to facilitate in the adhesion of one or more coatings of chemical agents and/or one or more coatings of polymer on the stent. The gas for the plasma etching can include carbon dioxide and/or other gasses. Once one or more surface regions of the stent have been treated, one or more coatings of polymer and/or biological agent can be applied to one or more regions of the stent. For instance, 1) one or more layers of porous or non-porous polymer can be coated on an outer and/or inner surface of the stent, 2) one or more layers of biological agent can be coated on an outer and/or inner surface of the stent, or 3) one or more layers of porous or non-porous polymer that includes one or more chemical agents can be coated on an outer and/or inner surface of the stent. The one or more layers of biological agent can be applied to the stent by a variety of techniques (e.g., dipping, rolling, brushing, spraying, particle atomization, etc.). One non-limiting coating technique is by an ultrasonic mist coating process wherein ultrasonic waves are used to break up the droplet of biological agent and form a mist of very fine droplets. These fine droplets have an average droplet diameter of about 0.1-3 microns. The fine droplet mist facilitates in the formation of a uniform coating thickness and can increase the coverage area on the stent.

In still yet another and/or alternative non-limiting aspect of the present invention, one or more portions of the stent can 1) include the same or different chemical agents, 2) include the same or different amount of one or more chemical agents, 3) include the same or different polymer coatings, 4) include the same or different coating thicknesses of one or more polymer coatings, 5) have one or more portions of the stent controllably release and/or uncontrollably release one or more chemical agents, and/or 6) have one or more portions of the stent controllably release one or more chemical agents and one or more portions of the stent uncontrollably release one or more chemical agents.

In yet another and/or alternative non-limiting aspect of the invention, the device can include a marker material that facilitates enabling the device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the device and/or be coated on one or more portions (flaring portion and/or body portion; at ends of device; at or near transition of body portion and flaring section; etc.) of the device. The location of the marker material can be on one or multiple locations on the device. The size of the one or more regions that include the marker material can be the same or different. The marker material can be spaced at defined distances from one another so as to form ruler-like markings on the device to facilitate in the positioning of the device in a body passageway. The marker material can be a rigid or flexible material. The marker material can be a biostable or biodegradable material. When the marker material is a rigid material, the marker material is typically formed of a metal material (e.g., metal band, metal plating, etc.); however, other or additional materials can be used. When the marker material is a flexible material, the marker material typically is formed of one or more polymers that are marker materials in-of-themselves and/or include one or more metal powders and/or metal compounds. In one non-limiting embodiment, the flexible marker material includes one or more metal powders in combinations with parylene, PLGA, POE, PGA, PLLA, PAA, PEG, chitosan and/or derivatives of one or more of these polymers.

In another and/or alternative non-limiting embodiment, the flexible marker material includes one or more metals and/or metal powders of aluminum, barium, bismuth, cobalt, copper, chromium, gold, iron, stainless steel, titanium, vanadium, nickel, zirconium, niobium, lead, molybdenum, platinum, yttrium, calcium, rare earth metals, magnesium, rhenium, zinc, silver, depleted radioactive elements, tantalum and/or tungsten; and/or compounds thereof. The marker material can be coated with a polymer protective material; however, this is not required. When the marker material is coated with a polymer protective material, the polymer coating can be used to 1) at least partially insulate the marker material from body fluids, 2) facilitate in retaining the marker material on the device, 3) at least partially shielding the marker material from damage during a medical procedure and/or 4) provide a desired surface profile on the device. As can be appreciated, the polymer coating can have other or additional uses. The polymer protective coating can be a biostable polymer or a biodegradable polymer (e.g., degrades and/or is absorbed). The coating thickness of the protective coating polymer material, when used, is typically less than about 300 microns; however, other thickness can be used. In one non-limiting embodiment, the protective coating materials include parylene, PLGA, POE, PGA, PLLA, PAA, PEG, chitosan and/or copolymers, blends, and/or composites of above and/or derivatives of one or more of these polymers.

In still another and/or alternative aspect of the invention, the stent can be an expandable device that can be expanded by use of another device (e.g., balloon, etc.) and/or is self expanding. The expandable stent can be fabricated from a material that has no or substantially no shape memory characteristics or can be fabricated from a material having shape-memory characteristics.

In a further and/or alternative non-limiting aspect of the present invention, the device or one or more regions of the device can be constructed by use of one or more microfabrication and/or micromachining technology used in creating Micro-Electro-Mechanical Systems (MEMS, e.g., micro-machining, laser micro machining, micro-molding, etc.); however, other or additional manufacturing techniques can be used. The device can include one or more surface structures (e.g., pore, channel, pit, rib, slot, notch, bump, teeth, well, hole, groove, etc.). These structures can be at least partially formed by MEMS technology and/or other types of technology. The device can include one or more micro-structures (e.g., micro-needle, micro-pore, micro-cylinder, micro-cone, micro-pyramid, micro-tube, microparallelopiped, micro-prism, micro-hemisphere, teeth, rib, ridge, ratchet, hinge, zipper, zip-tie like structure, etc.) on the inner, outer, or edge surface of the device. Non-limiting examples of structures that can be formed on the devices such as stent, graft, and/or other suitable devices are illustrated in United States Patent Publication Nos. 2004/0093076 and 2004/0093077, which are incorporated herein by reference. Typically, the micro-structures, when formed, extend from or into the outer surface no more than about 1000 microns, and more typically less than about 1000 microns; however, other sizes can be used. The micro-structures can be clustered together or disbursed throughout the surface of the device. Similar shaped and/or sized micro-structures and/or surface structures can be used, or different shaped and/or sized microstructures can be used. When one or more surface structures and/or micro-structures are designed to extend from the outer and/or inner surface of the device, the one or more surface structures and/or micro-structures can be formed in the extended position and/or be designed so as to extend from the device during and/or after deployment of the device in a treatment area. The micro-structures and/or surface structures can be designed to contain one or more agents and/or be connected to a passageway, cavity, etc. containing one or more agents; however, this is not required. The one or more surface structures and/or micro-structures can be used to engage and/or penetrate surrounding tissue or organs once the device has been positioned on and/or in a patient; however, this is not required. In another further and/or alternative non-limiting aspect of the present invention, the micro-structures and/or surface structures can be design to modify surface friction between the device and/or additional devices. For example, micro-structures and/or surface structures created on the inner surface of the device may be used to increase retention of a stent, graft, and/or other suitable device on a delivery catheter. In another further and/or alternative non-limiting aspect of the present invention, the micro-structures and/or surface structures can be design to create a system of undulations and/or crevasses used to facilitate growth of tissue. In one non-limiting aspect, the micro-structures and/or surface structures can be created on a film that could further be rolled into a shunt for neural regeneration, where the micro-structures and/or surface structures can provide a lattice to support and/or facilitate nerve growth. The one or more surface structures and/or micro-structures can be used to facilitate in forming or maintaining a shape of a device (i.e., see devices in United States Patent Publication Nos. 2004/0093076 and 2004/0093077). The one or more surface structures and/or micro-structures can be at least partially formed by MEMS technology; however, this is not required. In one non-limiting embodiment, the one or more surface structures and/or microstructures can be at least partially formed of an agent, polymer, agent polymer mixture, and/or layering of polymer and agent. One or more of the surface structures and/or micro-structures can include one or more internal passageways that can include one or more materials (e.g., agent, polymer, etc.); however, this is not required. In another further and/or alternative non-limiting aspect of the present invention, one or more internal passageways can be either connected and/or separated in part. The one or more surface structures and/or micro-structures can be formed by a variety of processes (e.g., machining, chemical modifications, chemical reactions, MEMS technology, etching, laser cutting, etc.). The one or more coatings and/or one or more surface structures and/or micro-structures of the device can be used for a variety of purposes such as, but not limited to, 1) increasing the bonding and/or adhesion of one or more agents, adhesives, marker materials and/or polymers to the device, 2) changing the appearance or surface characteristics of the device, and/or 3) controlling the release rate of one or more agents. The one or more microstructures and/or surface structures can be biostable, biodegradable, etc. One or more regions of the device that are at least partially formed by MEMS technology can be biostable, biodegradable, etc. The device or one or more regions of the device can be at least partially covered and/or filled with a protective material so as to at least partially protect one or more regions of the device, and/or one or more microstructures and/or surface structures on the device from damage. One or more regions of the device, and/or one or more micro-structures and/or surface structures on the device can be damaged when the device is 1) packaged and/or stored, 2) unpackaged, 3) connected to and/or otherwise secured and/or placed on another device, 4) inserted into a treatment area, 5) handled by a user, and/or 6) form a barrier between one or more micro-structures and/or surface structures and fluids in the body passageway. As can be appreciated, the device can be damaged in other or additional ways. The protective material can be used to protect the device and one or more micro-structures and/or surface structures from such damage. The protective material can include one or more polymers previously identified above. The protective material can be 1) biostable and/or biodegradable and/or 2) porous and/or non-porous. In one non-limiting design, the polymer is at least partially biodegradable so as to at least partially expose one or more micro-structure and/or surface structure to the environment after the device has been at least partially inserted into a treatment area. In another and/or additional non-limiting design, the protective material includes, but is not limited to, sugar (e.g., glucose, fructose, sucrose, etc.), carbohydrate compound, salt (e.g., NaCl, etc.), parylene, PLGA, POE, PGA, PLLA, PAA, PEG, chitosan and/or copolymers, blends, and/or composites of above and/or derivatives of one or more of these polymers; however, other and/or additional materials can be used. In still another and/or additional non-limiting design, the thickness of the protective material is generally less than about 300 microns, and typically less than about 150 microns; however, other thicknesses can be used depending upon the material chose of the protective material. The protective material can be coated by one or more mechanisms previously described herein.

In still yet another and/or alternative non-limiting aspect of the present invention, the device can include and/or be used with a physical hindrance. The physical hindrance can include, but is not limited to, an adhesive, a sheath, a magnet, tape, wire, string, etc. The physical hindrance can be used to 1) physically retain one or more regions of the device in a particular form or profile, 2) physically retain the device on a particular deployment device, 3) protect one or more surface structures and/or micro-structures on the device, and/or 4) form a barrier between one or more surface regions, surface structures and/or microstructures on the device and the fluids in a body passageway. As can be appreciated, the physical hindrance can have other and/or additional functions. The physical hindrance is typically a biodegradable material; however, a biostable material can be used. The physical hindrance can be designed to withstand sterilization of the device; however, this is not required. The physical hindrance can be applied to, included in and/or be used in conjunction with one or more devices. Additionally or alternatively, the physical hindrance can be designed to be used with and/or in conjunction with a device for a limited period of time and then 1) disengage from the device after the device has been partially or fully deployed and/or 2) dissolve and/or degrade during and/or after the device has been partially or fully deployed; however, this is not required. Additionally or alternatively, the physical hindrance can be designed and be formulated to be temporarily used with a device to facilitate in the deployment of the device; however, this is not required. In one non-limiting use of the physical hindrance, the physical hindrance is designed or formulated to at least partially secure a device to another device that is used to at least partially transport the device to a location for treatment. In another and/or alternative nonlimiting use of the physical hindrance, the physical hindrance is designed or formulated to at least partially maintain the device in a particular shape or form until the device is at least partially positioned in a treatment location. In still another and/or alternative nonlimiting use of the physical hindrance, the physical hindrance is designed or formulated to at least partially maintain and/or secure one type of device to another type of medical instrument or device until the device is at least partially positioned in a treatment location. The physical hindrance can also or alternatively be designed and formulated to be used with a device to facilitate in the use of the device. In one non-limiting use of the physical hindrance, when in the form of an adhesive, can be formulated to at least partially secure a device to a treatment area so as to facilitate in maintaining the device at the treatment area. For instance, the physical hindrance can be used in such use to facilitate in maintaining a device on or at a treatment area until the device is properly secured to the treatment area by sutures, stitches, screws, nails, rod, etc; however, this is not required. Additionally or alternatively, the physical hindrance can be used to facilitate in maintaining a device on or at a treatment area until the device has partially or fully accomplished its objective. The physical hindrance is typically a biocompatible material so as to not cause unanticipated adverse effects when properly used. The physical hindrance can be biostable or biodegradable (e.g., degrades and/or is absorbed, etc.). When the physical hindrance includes or is one or more adhesives, the one or more adhesives can be applied to the device by, but is not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition on the device. The physical hindrance can also or alternatively form at least a part of the device. One or more regions and/or surfaces of a device can also or alternatively include the physical hindrance. The physical hindrance can include one or more agents and/or other materials (e.g., marker material, polymer, etc.); however, this is not required. When the physical hindrance is or includes an adhesive, the adhesive can be formulated to controllably release one or more agents in the adhesive and/or coated on and/or contained within the device; however, this is not required. The adhesive can also or alternatively control the release of one or more agents located on and/or contained in the device by forming a penetrable or non-penetrable barrier to such agents; however, this is not required. The adhesive can include and/or be mixed with one or more polymers; however, this is not required. The one or more polymers can be used to 1) control the time of adhesion provided by said adhesive, 2) control the rate of degradation of the adhesive, and/or 3) control the rate of release of one or more agents from the adhesive and/or diffusing or penetrating through the adhesive layer; however, this is not required. When the physical hindrance includes a sheath, the sheath can be designed to partially or fully encircle the device. The sheath can be designed to be physically removed from the device after the device is deployed to a treatment area; however, this is not required. The sheath can be formed of a biodegradable material that at least partially degrades over time to at least partially expose one or more surface regions, micro-structures and/or surface structures of the device; however, this is not required. The sheath can include and/or be at least partially coated with one or more biological agents. The sheath includes one or more polymers; however, this is not required. The one or more polymers can be used for a variety of reasons such as, but not limited to, 1) forming a portion of the sheath, 2) improving a physical property of the sheath (e.g., improve strength, improve durability, improve biocompatibility, reduce friction, etc.), and/or 3 at least partially controlling a release rate of one or more agents from the sheath. As can be appreciated, the one or more polymers can have other or additional uses on the sheath.

In still a further and/or alternative non-limiting aspect of the present invention, the stent can be fully or partially formed of a base material that has biostable or bioabsorbable properties. The stent can be at least partially formed of one or more polymers, chemical agents, metals (e.g., aluminum, barium, bismuth, calcium, carbon, cobalt, copper, chromium, depleted radioactive elements, gold, iron, lead, molybdenum, magnesium, nickel, niobium, platinum, rare earth metals, rhenium, silver, tantalum, titanium, tungsten, vanadium, yttrium, zinc, zirconium, and/or alloys thereof (e.g., stainless steel, nitinol, Cr—Co, Mo—Re, Ta—W, Mg—Zr, Mg—Zn, brass, etc.)), ceramics, and/or fiber reinforced materials (e.g., carbon fiber material, fiberglass, etc.). The stent generally includes one or more materials that impart the desired properties to the stent so as to withstand the manufacturing process that is needed to produce the stent. These manufacturing processes can include, but are not limited to, laser cutting, etching, grinding, water cutting, spark erosion, crimping, annealing, drawing, pilgering, electroplating, electro-polishing, chemical polishing, ion beam deposition or implantation, sputter coating, vacuum deposition, etc.

In still a further and/or alternative non-limiting aspect of the present invention, the stent can be fully or partially formed of a base material that is at least partially made of a novel metal alloy having improved properties as compared to past stents that were form of stainless steel, or cobalt-chromium alloys. The novel metal alloy used to at least partially form the stent can improve one or more properties (e.g., strength, durability, hardness, biostability, bendability, coefficient of friction, radial strength, flexibility, tensile strength, longitudinal lengthening, stress-strain properties, improved recoil properties, radiopacity, heat sensitivity, biocompatibility, etc.) of such stent. These one or more physical properties of the novel metal alloy can be achieved in the stent without increasing the bulk, volume or weight of the stent, and in some instances can be obtained even when the volume, bulk and/or weight of the stent is reduced as compared to stents that are at least partially formed from traditional stainless steel or cobalt and chromium alloy materials. The novel metal alloy that is used to at least partially form the stent can thus 1) increase the radiopacity of the stent, 2) increase the radial strength of the stent, 3) increase the tensile strength of the stent, 4) improve the stress-strain properties of the stent, 5) improve the crimping and/or expansion properties of the stent, 6) improve the bendability and/or flexibility of the stent, 7) improve the strength and/or durability of the stent, 8) increase the hardness of the stent, 9) improve the longitudinal lengthening properties of the stent, 10) improved recoil properties of the stent, 11) improve the friction coefficient of the stent, 12) improve the heat sensitivity properties of the stent, 13) improve the biostability and/or biocompatibility properties of the stent, and/or 14) enable smaller, thinner and/or lighter weight stents to be made. It is believed that a smaller, thinner and/or lighter weight stent such as, but not limited to a stent, can be inserted in a body passageway and result in a decreased incidence of thrombosis. It is believed that such a stent will result in a less adverse response by the body when the stent is inserted in the body passageway. As such, the stent can be used without any biological agent included in, contained in, and/or coated on the stent and still result in a reduction in the incidence of thrombosis. As such, the need for extended use of body wide aggressive anti-platelet and/or anti-coagulation therapy after the stent has been inserted in the treatment area can be reduced or eliminated by use of the novel alloy.

In one non-limiting aspect of the present invention, a stent that can include the novel metal alloy is a stent for use in a body passageway; however, it can be appreciated that other types of stents could be at least partially formed from the novel metal alloy. As used herein, the term “body passageway” is defined to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, etc.). The techniques employed to deliver the stent to a treatment area include, but are not limited to, angioplasty, vascular anastomoses, interventional procedures, and any combinations thereof. For vascular applications, the term “body passageway” primarily refers to blood vessels and chambers in the heart. The stent can be an expandable stent that is expandable by a balloon and/or other means. The stent can have many shapes and forms. Such shapes can include, but are not limited to, stents disclosed in U.S. Pat. Nos. 6,206,916 and 6,436,133; and all the prior art cited in these patents. These various designs and configurations of stents in such patents are incorporated herein by reference.

In another and/or alternative non-limiting aspect of the present invention, the stent is generally designed to include at least about 25 weight percent of the novel metal alloy; however, this is not required. In one non-limiting embodiment of the invention, the stent includes at least about 40 weight percent of the novel metal alloy. In another and/or alternative non-limiting embodiment of the invention, the stent includes at least about 50 weight percent of the novel metal alloy. In still another and/or alternative non-limiting embodiment of the invention, the stent includes at least about 60 weight percent of the novel metal alloy. In yet another and/or alternative non-limiting embodiment of the invention, the stent includes at least about 70 weight percent of the novel metal alloy. In still yet another and/or alternative non-limiting embodiment of the invention, the stent includes at least about 85 weight percent of the novel metal alloy. In a further and/or alternative non-limiting embodiment of the invention, the stent includes at least about 90 weight percent of the novel metal alloy. In still a further and/or alternative non-limiting embodiment of the invention, the stent includes at least about 95 weight percent of the novel metal alloy. In yet a further and/or alternative non-limiting embodiment of the invention, the stent includes about 100 weight percent of the novel metal alloy.

In still another and/or alternative non-limiting aspect of the present invention, the novel metal alloy that is used to form all or part of the stent 1) is not clad, metal sprayed, plated and/or formed (e.g., cold worked, hot worked, etc.) onto another metal, or 2) does not have another metal or metal alloy metal sprayed, plated, clad and/or formed onto the novel metal alloy. It will be appreciated that in some applications, the novel metal alloy of the present invention may be clad, metal sprayed, plated and/or formed onto another metal, or another metal or metal alloy may be plated, metal sprayed, clad and/or formed onto the novel metal alloy when forming all or a portion of a stent.

In yet another and/or alternative non-limiting aspect of the present invention, the novel metal alloy that is used to form all or a portion of the stent includes rhenium and molybdenum. The novel metal alloy can include one or more other metals such as, but not limited to, boron, calcium, chromium, cobalt, copper, gold, iron, lead, magnesium, manganese, mercury, nickel, niobium, platinum, rare earth metals, silicon, silver, sulfur, tantalum, tin, titanium, tungsten, yttrium, zinc, zirconium, and/or alloys thereof.

In still another and/or alternative non-limiting aspect of the present invention, the novel metal alloy that is used to form all or a portion of the stent is a novel metal alloy that includes at least about 90 weight percent molybdenum and rhenium. In one non-limiting composition, the content of molybdenum and rhenium in the novel metal alloy is at least about 95 weight percent. In another and/or alternative non-limiting composition, the content of molybdenum and rhenium in the novel metal alloy is at least about 97 weight percent. In still another and/or alternative non-limiting composition, the content of molybdenum and rhenium in the novel metal alloy is at least about 98 weight percent. In yet another and/or alternative non-limiting composition, the content of molybdenum and rhenium in the novel metal alloy is at least about 99 weight percent. In still yet another and/or alternative non-limiting composition, the content of molybdenum and rhenium in the novel metal alloy is at least about 99.5 weight percent. In a further one non-limiting composition, the content of molybdenum and rhenium in the novel metal alloy is at least about 99.9 weight percent. In still a further and/or alternative non-limiting composition, the content of molybdenum and rhenium in the novel metal alloy is at least about 99.95 weight percent. In yet a further and/or alternative non-limiting composition, the content of molybdenum and rhenium in the novel metal alloy is at least about 99.99 weight percent. As can be appreciated, other weight percentages of the rhenium and molybdenum content of the novel metal alloy can be used. In one non-limiting composition, the purity level of the novel metal alloy is such so as to produce a solid solution of the novel metal alloy. A solid solution or homogeneous solution is defined as a metal alloy that includes two or more primary metals and the combined weight percent of the primary metals is at least about 95 weight percent, typically at least about 99 weight percent, more typically at least about 99.5 weight percent, even more typically at least about 99.8 weight percent, and still even more typically at least about 99.9 weight percent. A primary metal is a metal component of the metal alloy that is not a metal impurity. A solid solution of a novel metal alloy that includes rhenium and molybdenum as the primary metals is an alloy that includes at least about 95-99 weight percent rhenium and molybdenum. It is believed that a purity level of less than 95 weight percent molybdenum and rhenium adversely affects one or more physical properties of the metal alloy that are useful or desired in forming and/or using a stent. In one embodiment of the invention, the rhenium content of the novel metal alloy in accordance with the present invention is at least about 40 weight percent. In one non-limiting composition, the rhenium content of the novel metal alloy is at least about 45 weight percent. In still another and/or alternative non-limiting composition, the rhenium content of the novel metal alloy is about 45-50 weight percent. In yet another and/or alternative non-limiting composition, the rhenium content of the novel metal alloy is about 47-48 weight percent. In still yet another and/or alternative non-limiting composition, the rhenium content of the novel metal alloy is about 47.6-49.5 weight percent. In still another and/or alternative non-limiting composition, the rhenium content of the novel metal alloy is about 47.15-47.5 weight percent. As can be appreciated, other weight percentages of the rhenium content of the novel metal alloy can be used. In another and/or alternative embodiment of the invention, the molybdenum content of the novel metal alloy in accordance with the present invention is at least about 40 weight percent. In one non-limiting composition, the molybdenum content of the novel metal alloy is at least about 45 weight percent. In another and/or alternative non-limiting composition, the molybdenum content of the novel metal alloy is at least about 50 weight percent. In still another and/or alternative non-limiting composition, the molybdenum content of the novel metal alloy is about 50-60 percent. In yet another and/or alternative non-limiting composition, the molybdenum content of the novel metal alloy is about 50-56 weight percent. As can be appreciated, other weight percentages of the molybdenum content of the novel metal alloy can be used.

In still yet another and/or alternative non-limiting aspect of the present invention, the novel metal alloy that is used to form all or a portion of the stent is a novel metal alloy that includes at least about 90 weight percent molybdenum and rhenium, and at least one additional metal which includes titanium, yttrium, and/or zirconium. The addition of controlled amounts of titanium, yttrium, and/or zirconium to the molybdenum and rhenium alloy has been found to form a metal alloy that has improved physical properties over a metal alloy that principally includes molybdenum and rhenium. For instance, the addition of controlled amounts of titanium, yttrium, and/or zirconium to the molybdenum and rhenium alloy can result in 1) an increase in yield strength of the alloy as compared to a metal alloy that principally includes molybdenum and rhenium, 2) an increase in tensile elongation of the alloy as compared to a metal alloy that principally includes molybdenum and rhenium, 3) an increase in ductility of the alloy as compared to a metal alloy that principally includes molybdenum and rhenium, 4) a reduction in grain size of the alloy as compared to a metal alloy that principally includes molybdenum and rhenium, 5) a reduction in the amount of free carbon, oxygen and/or nitrogen in the alloy as compared to a metal alloy that principally includes molybdenum and rhenium, and/or 6) a reduction in the tendency of the alloy to form micro-cracks during the forming of the alloy into a stent as compared to the forming of a stent from a metal alloy that principally includes molybdenum and rhenium. In one non-limiting composition, the content of molybdenum and rhenium and the at least one additional metal in the novel metal alloy is at least about 90 weight percent. In another and/or alternative non-limiting composition, the content of molybdenum and rhenium and the at least one additional metal in the novel metal alloy is at least about 95 weight percent. In still another and/or alternative non-limiting composition, the content of molybdenum and rhenium and the at least one additional metal in the novel metal alloy is at least about 98 weight percent. In yet another and/or alternative non-limiting composition, the content of molybdenum and rhenium and the at least one additional metal in the novel metal alloy is at least about 99 weight percent. In still yet another and/or alternative non-limiting composition, the content of molybdenum and rhenium and the at least one additional metal in the novel metal alloy is at least about 99.5 weight percent. In a further one non-limiting composition, the content of molybdenum and rhenium and the at least one additional metal in the novel metal alloy is at least about 99.9 weight percent. In still a further and/or alternative non-limiting composition, the content of molybdenum and rhenium and the at least one additional metal in the novel metal alloy is at least about 99.95 weight percent. In yet a further and/or alternative non-limiting composition, the content of molybdenum and rhenium and the at least one additional metal in the novel metal alloy is at least about 99.99 weight percent. As can be appreciated, other weight percentages of the content of molybdenum and rhenium and the at least one additional metal in the novel metal alloy can be used. In one non-limiting composition, the purity level of the novel metal alloy is such so as to produce a solid solution of a rhenium and molybdenum and the at least one additional metal. A solid solution of a novel metal alloy that includes rhenium and molybdenum and the at least one additional metal of titanium, yttrium and/or zirconium as the primary metals is an alloy that includes at least about 95-99 weight percent rhenium and molybdenum and the at least one additional metal. It is believed that a purity level of less than 95 weight percent molybdenum and rhenium and the at least one additional metal adversely affects one or more physical properties of the metal alloy that are useful or desired in forming and/or using a stent. In one embodiment of the invention, the rhenium content of the novel metal alloy in accordance with the present invention is at least about 40 weight percent. In one non-limiting composition, the rhenium content of the novel metal alloy is at least about 45 weight percent. In still another and/or alternative non-limiting composition, the rhenium content of the novel metal alloy is about 45-50 weight percent. In yet another and/or alternative non-limiting composition, the rhenium content of the novel metal alloy is about 47-48 weight percent. As can be appreciated, other weight percentages of the rhenium content of the novel metal alloy can be used. In another and/or alternative embodiment of the invention, the molybdenum content of the novel metal alloy is at least about 40 weight percent. In one non-limiting composition, the molybdenum content of the novel metal alloy is at least about 45 weight percent. In another and/or alternative non-limiting composition, the molybdenum content of the novel metal alloy is at least about 50 weight percent. In still another and/or alternative non-limiting composition, the molybdenum content of the novel metal alloy is about 50-60 percent. In yet another and/or alternative non-limiting composition, the molybdenum content of the novel metal alloy is about 50-56 weight percent. As can be appreciated, other weight percentages of the molybdenum content of the novel metal alloy can be used. The combined content of titanium, yttrium and zirconium in the novel metal alloy is less than about 5 weight percent, typically no more than about 1 weight percent, and more typically no more than about 0.5 weight percent. A higher weight percent content of titanium, yttrium and/or zirconium in the novel metal alloy can begin to adversely affect the brittleness of the novel metal alloy. When titanium is included in the novel metal alloy, the titanium content is typically less than about 1 weight percent, more typically less than about 0.6 weight percent, even more typically about 0.05-0.5 weight percent, still even more typically about 0.1-0.5 weight percent. As can be appreciated, other weight percentages of the titanium content of the novel metal alloy can be used. When zirconium is included in the novel metal alloy, the zirconium content is typically less than about 0.5 weight percent, more typically less than about 0.3 weight percent, even more typically about 0.01-0.25 weight percent, still even more typically about 0.05-0.25 weight percent. As can be appreciated, other weight percentages of the zirconium content of the novel metal alloy can be used. When titanium and zirconium are included in the novel metal alloy, the weight ratio of titanium to zirconium is about 1-10:1, typically about 1.5-5:1, and more typically about 1.75-2.5:1. When yttrium is included in the novel metal alloy, the yttrium content is typically less than about 0.3 weight percent, more typically less than about 0.2 weight percent, and even more typically about 0.01-0.1 weight percent. As can be appreciated, other weight percentages of the yttrium content of the novel metal alloy can be used. The inclusion of titanium, yttrium and/or zirconium in the novel metal alloy is believed to result in a reduction of oxygen trapped in the solid solution of the novel metal alloy. The reduction of trapped oxygen enables the formation of a smaller grain size in the novel metal alloy and/or an increase in the ductility of the novel metal alloy. The reduction of trapped oxygen in the novel metal alloy can also increase the yield strength of the novel metal alloy as compared to alloys of only molybdenum and rhenium (i.e., 2-10% increase). The inclusion of titanium, yttrium and/or zirconium in the novel metal alloy is also believed to cause a reduction in the trapped free carbon in the novel metal alloy. The inclusion of titanium, yttrium and/or zirconium in the novel metal alloy is believed to form carbides with the free carbon in the novel metal alloy. This carbide formation is also believed to improve the ductility of the novel metal alloy and to also reduce the incidence of cracking during the forming of the metal alloy into a stent (e.g., stent, etc.). As such, the novel metal alloy exhibits increased tensile elongation as compared to alloys of only molybdenum and rhenium (i.e., 1-8% increase). The inclusion of titanium, yttrium and/or zirconium in the novel metal alloy is also believed to cause a reduction in the trapped free nitrogen in the novel metal alloy. The inclusion of titanium, yttrium and/or zirconium in the novel metal alloy is believed to form carbo-nitrides with the free carbon and free nitrogen in the novel metal alloy. This carbo-nitride formation is also believed to improve the ductility of the novel metal alloy and to also reduce the incidence of cracking during the forming of the metal alloy into a stent. As such, the novel metal alloy exhibits increased tensile elongation as compared to alloys of only molybdenum and rhenium (i.e., 1-8% increase). The reduction in the amount of free carbon, oxygen and/or nitrogen in the novel metal alloy is also believed to increase the density of the novel metal alloy (i.e., 1-5% increase). The formation of carbides, carbo-nitrides, and/or oxides in the novel metal alloy results in the formation of dispersed second phase particles in the novel metal alloy, thereby facilitating in the formation of small grain sizes in the metal alloy.

In still another and/or alternative non-limiting aspect of the present invention, the novel metal alloy includes less than about 5 weight percent other metals and/or impurities. A high purity level of the novel metal alloy results in the formation of a more homogeneous alloy, which in turn results in a more uniform density throughout the novel metal alloy, and also results in the desired yield and ultimate tensile strengths of the novel metal alloy. The density of the novel metal alloy is generally at least about 12 gm/cc, and typically at least about 13-13.5 gm/cc. This substantially uniform high density of the novel metal alloy significantly improves the radiopacity of the novel metal alloy. In one non-limiting composition, the novel metal alloy includes less than about 1 weight percent other metals and/or impurities. In another and/or alternative non-limiting composition, the novel metal alloy includes less than about 0.5 weight percent other metals and/or impurities. In still another and/or alternative non-limiting composition, the novel metal alloy includes less than about 0.4 weight percent other metals and/or impurities. In yet another and/or alternative non-limiting composition, the novel metal alloy includes less than about 0.2 weight percent other metals and/or impurities. In still yet another and/or alternative non-limiting composition, the novel metal alloy includes less than about 0.1 weight percent other metals and/or impurities. In still another and/or alternative non-limiting composition, the novel metal alloy includes less than about 0.08 weight percent other metals and/or impurities. In yet another and/or alternative non-limiting composition, the novel metal alloy includes less than about 0.06 weight percent other metals and/or impurities. In a further and/or alternative non-limiting composition, the novel metal alloy includes less than about 0.05 weight percent other metals and/or impurities. In still a further and/or alternative non-limiting composition, the novel metal alloy includes less than about 0.02 weight percent other metals and/or impurities. In yet a further and/or alternative non-limiting composition, the novel metal alloy includes less than about 0.01 weight percent other metals and/or impurities. As can be appreciated, other weight percentages of the amount of other metals and/or impurities in the novel metal alloy can exist.

In yet another and/or alternative non-limiting aspect of the present invention, the novel metal alloy includes a certain amount of carbon and oxygen. These two elements have been found to affect the forming properties and brittleness of the novel metal alloy. The controlled atomic ratio of carbon and oxygen in the novel metal alloy also can be used to minimize the tendency of the novel metal alloy to form micro-cracks during the forming of the novel metal alloy into a stent, and/or during the use and/or expansion of the stent in a body passageway. The control of the atomic ratio of carbon to oxygen in the novel metal alloy allows for the redistribution of oxygen in the metal alloy so as to minimize the tendency of micro-cracking in the novel metal alloy during the forming of the novel metal alloy into a stent, and/or during the use and/or expansion of the stent in a body passageway. The atomic ratio of carbon to oxygen in the alloy is believed to be important to minimize the tendency of micro-cracking in the novel metal alloy, improve the degree of elongation of the novel metal alloy, both of which can affect one or more physical properties of the metal alloy that are useful or desired in forming and/or using the stent. It was previously believed by applicants that a carbon to oxygen atomic ratio of less than about 2:1 would adversely affect the properties of a stent such as, but not limited to a stent. Upon further investigation, it has been found that a stent when exposed to body temperatures can be formed of the novel metal alloy with a carbon to oxygen atomic ratio that is less than about 2:1; however, it is still believed that the properties of the stent are better when the carbon to oxygen atomic ratio is greater than about 2:1. It is believed that for certain applications of the novel metal alloy when operating in temperatures of about 40-120° F. and that the oxygen content is below a certain amount, the carbon to oxygen atomic ratio can be as low as about 0.2:1. In one non-limiting formulation, the carbon to oxygen atomic ratio in the novel metal alloy is generally at least about 0.4:1 (i.e., weight ratio of about 0.3:1). In another non-limiting formulation, the carbon to oxygen atomic ratio in the novel metal alloy is generally at least about 0.5:1 (i.e., weight ratio of about 0.375:1). In still another non-limiting formulation, the carbon to oxygen atomic ratio in the novel metal alloy is generally at least about 1:1 (i.e., weight ratio of about 0.75:1). In yet another non-limiting formulation, the carbon to oxygen atomic ratio in the novel metal alloy is generally at least about 2:1 (i.e., weight ratio of about 1.5:1). In still yet another non-limiting formulation, the carbon to oxygen atomic ratio in the novel metal alloy is generally at least about 2.5:1 (i.e., weight ratio of about 1.88:1). In still another non-limiting formulation, the carbon to oxygen atomic ratio in the novel metal alloy is generally at least about 3:1 (i.e., weight ratio of about 2.25:1). In yet another non-limiting formulation, the carbon to oxygen atomic ratio in the novel metal alloy is generally at least about 4:1 (i.e., weight ratio of about 3:1). In still yet another non-limiting formulation, the carbon to oxygen atomic ratio in the novel metal alloy is generally at least about 5:1 (i.e., weight ratio of about 3.75:1). In still another non-limiting formulation, the carbon to oxygen atomic ratio in the novel metal alloy is generally about 2.5-50:1 (i.e., weight ratio of about 1.88-37.54:1). In a further non-limiting formulation, the carbon to oxygen atomic ratio in the novel metal alloy is generally about 2.5-20:1 (i.e., weight ratio of about 1.88-15:1). In a further non-limiting formulation, the carbon to oxygen atomic ratio in the novel metal alloy is generally about 2.5-13.3:1 (i.e., weight ratio of about 1.88-10:1). In still a further non-limiting formulation, the carbon to oxygen atomic ratio in the novel metal alloy is generally about 2.5-10:1 (i.e., weight ratio of about 1.88-7.5:1). In yet a further non-limiting formulation, the carbon to oxygen atomic ratio in the novel metal alloy is generally about 2.5-5:1 (i.e., weight ratio of about 1.88-3.75:1). As can be appreciated, other atomic ratios of the carbon to oxygen in the novel metal alloy can be used. The carbon to oxygen ratio can be adjusted by intentionally adding carbon to the novel metal alloy until the desired carbon to oxygen ratio is obtained. Typically the carbon content of the novel metal alloy is less than about 0.2 weight percent. Carbon contents that are too large can adversely affect the physical properties of the novel metal alloy. In one non-limiting formulation, the carbon content of the novel metal alloy is less than about 0.1 weight percent of the novel metal alloy. In another non-limiting formulation, the carbon content of the novel metal alloy is less than about 0.05 weight percent of the novel metal alloy. In still another non-limiting formulation, the carbon content of the novel metal alloy is less than about 0.04 weight percent of the novel metal alloy. When carbon is not intentionally added to the novel metal alloy, the novel metal alloy can include up to about 150 ppm carbon, typically up to about 100 ppm carbon, and more typically less than about 50 ppm carbon. The oxygen content of the novel metal alloy can vary depending on the processing parameters used to form the novel metal alloy. Generally, the oxygen content is to be maintained at very low levels. In one non-limiting formulation, the oxygen content is less than about 0.1 weight percent of the novel metal alloy. In another non-limiting formulation, the oxygen content is less than about 0.05 weight percent of the novel metal alloy. In still another non-limiting formulation, the oxygen content is less than about 0.04 weight percent of the novel metal alloy. In yet another non-limiting formulation, the oxygen content is less than about 0.03 weight percent of the novel metal alloy. In still yet another non-limiting formulation, the novel metal alloy includes up to about 100 ppm oxygen. In a further non-limiting formulation, the novel metal alloy includes up to about 75 ppm oxygen. In still a further non-limiting formulation, the novel metal alloy includes up to about 50 ppm oxygen. In yet a further non-limiting formulation, the novel metal alloy includes up to about 30 ppm oxygen. In still yet a further non-limiting formulation, the novel metal alloy includes less than about 20 ppm oxygen. In yet a further non-limiting formulation, the novel metal alloy includes less than about 10 ppm oxygen. As can be appreciated, other amounts of carbon and/or oxygen in the novel metal alloy can exist. It is believed that the novel metal alloy will have a very low tendency to form micro-cracks during the formation of the stent and after the stent has been inserted into a patient by closely controlling the carbon to oxygen ration when the oxygen content exceed a certain amount in the novel metal alloy. In one non-limiting arrangement, the carbon to oxygen atomic ratio in the novel metal alloy is at least about 2.5:1 when the oxygen content is greater than about 100 ppm in the novel metal alloy.

In still yet another and/or alternative non-limiting aspect of the present invention, the novel metal alloy includes a controlled amount of nitrogen. Large amounts of nitrogen in the novel metal alloy can adversely affect the ductility of the novel metal alloy. This can in turn adversely affect the elongation properties of the novel metal alloy. A too high of nitrogen content in the novel metal alloy can begin to cause the ductility of the novel metal alloy to unacceptably decrease, thus adversely affect one or more physical properties of the metal alloy that are useful or desired in forming and/or using the stent. In one non-limiting formulation, the novel metal alloy includes less than about 0.001 weight percent nitrogen. In another non-limiting formulation, the novel metal alloy includes less than about 0.0008 weight percent nitrogen. In still another non-limiting formulation, the novel metal alloy includes less than about 0.0004 weight percent nitrogen. In yet another non-limiting formulation, the novel metal alloy includes less than about 30 ppm nitrogen. In still yet another non-limiting formulation, the novel metal alloy includes less than about 25 ppm nitrogen. In still another non-limiting formulation, the novel metal alloy includes less than about 10 ppm nitrogen. In yet another non-limiting formulation, the novel metal alloy includes less than about 5 ppm nitrogen. As can be appreciated, other amounts of nitrogen in the novel metal alloy can exist. The relationship of carbon, oxygen and nitrogen in the novel metal alloy is also believed to be important. It is believed that the nitrogen content should be less than the content of carbon or oxygen in the novel metal alloy. In one non-limiting formulation, the atomic ratio of carbon to nitrogen is at least about 2:1 (i.e., weight ratio of about 1.71:1). In another non-limiting formulation, the atomic ratio of carbon to nitrogen is at least about 3:1 (i.e., weight ratio of about 2.57:1). In still another non-limiting formulation, the atomic ratio of carbon to nitrogen is about 4-100:1 (i.e., weight ratio of about 3.43-85.7:1). In yet another non-limiting formulation, the atomic ratio of carbon to nitrogen is about 4-75:1 (i.e., weight ratio of about 3.43-64.3:1). In still another non-limiting formulation, the atomic ratio of carbon to nitrogen is about 4-50:1 (i.e., weight ratio of about 3.43-42.85:1). In yet another non-limiting formulation, the atomic ratio of carbon to nitrogen is about 4-35:1 (i.e., weight ratio of about 3.43-30:1). In still yet another non-limiting formulation, the atomic ratio of carbon to nitrogen is about 4-25:1 (Le., weight ratio of about 3.43-21.43:1). In a further non-limiting formulation, the atomic ratio of oxygen to nitrogen is at least about 1.2:1 (i.e., weight ratio of about 1.37:1). In another non-limiting formulation, the atomic ratio of oxygen to nitrogen is at least about 2:1 (i.e., weight ratio of about 2.28:1). In still another non-limiting formulation, the atomic ratio of oxygen to nitrogen is about 3-100:1 (i.e., weight ratio of about 3.42-114.2:1). In yet another non-limiting formulation, the atomic ratio of oxygen to nitrogen is at least about 3-75:1 (i.e., weight ratio of about 3.42-85.65:1). In still yet another non-limiting formulation, the atomic ratio of oxygen to nitrogen is at least about 3-55:1 (i.e., weight ratio of about 3.42-62.81:1). In yet another non-limiting formulation, the atomic ratio of oxygen to nitrogen is at least about 3-50:1 (i.e., weight ratio of about 3.42-57.1:1).

In a further and/or alternative non-limiting aspect of the present invention, the novel metal alloy has several physical properties that positively affect the stent when at least partially formed of the novel metal alloy. In one non-limiting embodiment of the invention, the average hardness of the novel metal alloy tube used to form the stent is generally at least about 60 (HRC) at 77° F. In one non-limiting aspect of this embodiment, the average hardness of the novel metal alloy tube used to form the stent is generally at least about 70 (HRC) at 77° F., and typically about 80-100 (HRC) at 77° F. In another and/or alternative non-limiting embodiment of the invention, the average ultimate tensile strength of the novel metal alloy used to form the stent is generally at least about 60 UTS (ksi). In non-limiting aspect of this embodiment, the average ultimate tensile strength of the novel metal alloy used to form the stent is generally at least about 70 UTS (ksi), typically about 80-150 UTS (ksi), and more typically about 100-150 UTS (ksi). In still another and/or alternative non-limiting embodiment of the invention, the average yield strength of the novel metal alloy used to form in the stent is at least about 70 ksi. In one non-limiting aspect of this embodiment, the average yield strength of the novel metal alloy used to form the stent is at least about 80 ksi, and typically about 100-140 (ksi). In yet another and/or alternative non-limiting embodiment of the invention, the average grain size of the novel metal alloy used to form the stent is greater than 5 ASTM (e.g., ASTM E 112-96). The small grain size of the novel metal alloy enables the stent to have the desired elongation and ductility properties that are useful in enabling the stent to be formed, crimped and/or expanded. In one non-limiting aspect of this embodiment, the average grain size of the novel metal alloy used to form the stent is about 5.2-10 ASTM, typically, about 5.5-9 ASTM, more typically about 6-9 ASTM, still more typically about 6-8 ASTM, even more typically, about 6-7 ASTM, and still even more typically about 6.5-7 ASTM. In still yet another and/or alternative non-limiting embodiment of the invention, the average tensile elongation of the novel metal alloy used to form the stent is at least about 25%. An average tensile elongation of at least 25% for the novel metal alloy is important to enable the stent to be properly expanded when positioned in the treatment area of a body passageway. A stent that does not have an average tensile elongation of at least about 25% can form micro-cracks and/or break during the forming, crimping and/or expansion of the stent. In one non-limiting aspect of this embodiment, the average tensile elongation of the novel metal alloy used to form the stent is about 25-35%. The unique combination of the rhenium content in the novel metal alloy in combination with achieving the desired purity and composition of the alloy and the desired gain size of the novel metal alloy results in 1) a stent having the desired high ductility at about room temperature, 2) a stent having the desired amount of tensile elongation, 3) a homogeneous or solid solution of a metal alloy having high radiopacity, 4) a reduction or prevention of microcrack formation and/or breaking of the metal alloy tube when the metal alloy tube is sized and/or cut to form the stent, 5) a reduction or prevention of microcrack formation and/or breaking of the stent when the stent is crimped onto a balloon and/or other type of stent for insertion into a body passageway, 6) a reduction or prevention of microcrack formation and/or breaking of the stent when the stent is bent and/or expanded in a body passageway, 7) a stent having the desired ultimate tensile strength and yield strength, 8) a stent that can have very thin wall thicknesses and still have the desired radial forces needed to retain the body passageway on an open state when the stent has been expanded, and/or 9) a stent that exhibits less recoil when the stent is crimped onto a delivery system and/or expanded in a body passageway.

In another and/or alternative non-limiting aspect of the present invention, the use of the novel metal alloy in the stent can increase the strength of the stent as compared with stainless steel or chromium-cobalt alloys, thus less quantity of novel metal alloy can be used in the stent to achieve similar strengths as compared to stents formed of different metals. As such, the resulting stent can be made smaller and less bulky by use of the novel metal alloy without sacrificing the strength and durability of the stent. Such a stent can have a smaller profile, thus can be inserted in smaller areas, openings and/or passageways. The novel metal alloy also can increase the radial strength of the stent. For instance, the thickness of the walls of the stent and/or the wires used to form the stent can be made thinner and achieve a similar or improved radial strength as compared with thicker walled stents formed of stainless steel or cobalt and chromium alloy. The novel metal alloy also can improve stress-strain properties, bendability and flexibility of the stent, thus increase the life of the stent. For instance, the stent can be used in regions that subject the stent to bending. Due to the improved physical properties of the stent from the novel metal alloy, the stent has improved resistance to fracturing in such frequent bending environments. In addition or alternatively, the improved bendability and flexibility of the stent due to the use of the novel metal alloy can enable the stent to be more easily inserted into a body passageway. The novel metal alloy can also reduce the degree of recoil during the crimping and/or expansion of the stent. For example, the stent better maintains its crimped form and/or better maintains its expanded form after expansion due to the use of the novel metal alloy. As such, when the stent is to be mounted onto a delivery device when the stent is crimped, the stent better maintains its smaller profile during the insertion of the stent in a body passageway. Also, the stent better maintains its expanded profile after expansion so as to facilitate in the success of the stent in the treatment area. In addition to the improved physical properties of the stent by use of the novel metal alloy, the novel metal alloy has improved radiopaque properties as compared to standard materials such as stainless steel or cobalt-chromium alloy, thus reducing or eliminating the need for using marker materials on the stent. For instance, the novel metal alloy is at least about 10-20% more radiopaque than stainless steel or cobalt-chromium alloy. Specifically, the novel metal alloy can be at least about 33% more radiopaque than cobalt-chromium alloy and at least about 41.5% more radiopaque than stainless steel.

In still yet another and/or alternative non-limiting aspect of the present invention, the stent that is at least partially formed from the novel metal alloy can be formed by a variety of manufacturing techniques. In one non-limiting embodiment of the invention, the stent can be formed from a rod or tube of the novel metal alloy. If a solid rod of the novel metal alloy is formed, the rod can be cut or drilled (e.g., gun drilled, EDM, etc.) to form a cavity or passageway partially or fully through the rod. The rod or tube can be cleaned, polished, annealed, drawn, etc. to obtain the desired cross-sectional area or diameter and/or wall thickness of the metal tube. After the metal tube has been formed to the desired cross-sectional area or diameter and wall thickness, the metal tube can be formed into a stent by a process such as, but not limited to, laser cutting, etching, etc. After the stent has been formed, the stent can be cleaned, polished, sterilized, etc. for final processing of the stent. As can be appreciated, other or additional process steps can be used to at least partially form the stent from the novel metal alloy.

One non-limiting object of the present invention is the provision of a stent that can be formed from conventional materials or include new materials having a lower ductility than conventional materials.

Another and/or additional non-limiting object of the present invention is the provision of a stent having improved procedural success rates.

Still another and/or additional non-limiting object of the present invention is the provision of a stent that is formed of a material that improves the physical properties of the stent.

Yet another and/or additional non-limiting object of the present invention is the provision of a stent that is simple and cost effective to manufacture.

Still yet another and/or additional non-limiting object of the present invention is the provision of a stent that allows for deformation to occur at at least one of the hinge points as well as along the length of at least one of the struts of the stent.

Another and/or additional non-limiting object of the present invention is the provision of a stent that reduces the maximum stress at the hinge point and distributing the stresses beyond at least one of the hinge points.

Still another and/or additional non-limiting object of the present invention is the provision of a stent that is more flexible.

Yet another and/or additional non-limiting object of the present invention is the provision of a stent that includes an undulating pattern along at least a portion of the length of one or more struts on the stent.

Still yet another and/or additional non-limiting object of the present invention is the provision of a stent that reduces the need for long articulations between the strut rings.

Another and/or additional non-limiting object of the present invention is the provision of a stent that includes more rings to be placed within a given length of the stent.

Still another and/or additional non-limiting object of the present invention is the provision of a stent reduces the open areas and improves the radial force of the stent.

Yet another and/or additional non-limiting object of the present invention is the provision of a stent that reduces the strain at at least one of the hinges between the struts.

Still yet another and/or additional non-limiting object of the present invention is the provision of a stent that reduces at least one of the strut widths along the length of at least one of the struts to cause it to bend at the narrowest region.

Another and/or additional non-limiting object of the present invention is the provision of a stent that reduces at least one of the connector widths along the length of at least one of the connectors thus causing it to bend at the narrowest region.

Still another and/or additional non-limiting object of the present invention is the provision of a stent that provides at least one of an undulating pattern along at least a portion of the length of one or more connectors.

A further and/or additional non-limiting object of the present invention is the provision of a stent that includes one or mire dimples and/or divots on at least a portion of one or more the struts on the stent.

A still further and/or alternative non-limiting object of the present invention is the provision of a stent that includes one or more chemical agents.

These and other advantages will become apparent to those skilled in the art upon the reading and following of this description taken together with the accompanying drawings.

### BRIEF DESCRIPTION OF THE DRAWINGS

Reference may now be made to the drawings, which illustrate various embodiments that the invention may take in physical form and in certain parts and arrangements of parts wherein:

FIG. 1 is a perspective view of a section of a stent in the form of an unexpanded stent which permits delivery of the stent into a body passageway in accordance with the present invention;

FIG. 2 is a plan view of the stent of FIG. 1 prior to being rolled into a tubular form;

FIG. 3 is an enlarged view of a portion of the stent of FIG. 1;

FIG. 4 is an enlarged view of a portion of a stent in accordance with the present invention;

FIG. 5 is an enlarged view of a portion of a stent in accordance with the present invention;

FIG. 6 is an enlarged view of a portion of a stent in accordance with the present invention;

FIG. 7 is an enlarged view of a portion of a stent in accordance with the present invention;

FIG. 8 is an enlarged view of a portion of a stent in accordance with the present invention;

FIG. 9 is an enlarged view of a portion of a stent in accordance with the present invention;

FIG. 10 is a is a plan view of the stent prior to being rolled into a tubular form in accordance with the present invention;

FIG. 11 is an enlarged view of a portion of a stent in accordance with the present invention;

FIG. 12 is an enlarged view of a portion of a stent in accordance with the present invention;

FIG. 13 is an enlarged view of a portion of a stent in accordance with the present invention; and,

FIG. 14 is an enlarged view of a portion of a stent in accordance with the present invention.

### DETAILED DESCRIPTION OF THE INVENTION

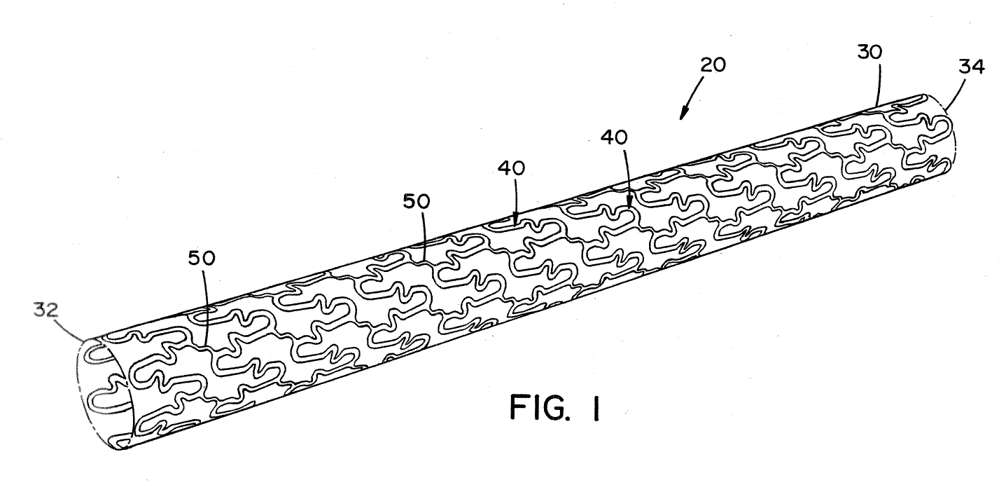

Referring now to the drawings wherein the showings are for the purpose of illustrating embodiments of the invention only and not for the purpose of limiting the same, FIGS. 1-10 disclose a stent in the form of a stent for use in a body passageway. The stent is particularly useful in the cardiovascular field; however, the stent can be used in other medical fields such as, but not limited to, orthopedic field, cardiology field, pulmonology field, urology field, nephrology field, gastroenterology field, gynecology field, otolaryngology field or other surgical fields. Additionally or alternatively, the stent is not limited to a stent, thus can be in the form of many other stents (e.g., a staple, an orthopedic implant, a valve, a vascular implant, a pacemaker, a spinal implant, a guide wire, nail, rod, screw, etc.).

The stent, when used for vascular applications, can be used to address various medical problems such as, but not limited to, restenosis, atherosclerosis, atherogenesis, angina, ischemic disease, congestive heart failure or pulmonary edema associated with acute myocardial infarction, atherosclerosis, thrombosis, controlling blood pressure in hypertension, platelet adhesion, platelet aggregation, smooth muscle cell proliferation, vascular complications, wounds, myocardial infarction, pulmonary thromboembolism, cerebral thromboembolism, thrombophlebitis, thrombocytopenia or bleeding disorders.

As illustrated in FIG. 1, stent 20 is in the form of an expandable stent that includes at least one tubular shaped body member 30 having a first end 32, a second end 34, and struts 40 and connectors 50 disposed between the first and second ends. As can be appreciated, the stent can be formed of a single body member or a plurality of body members connected together. Body member 30 has a first diameter which permits delivery of the body member into a body passageway. The first diameter of the body member is illustrated as substantially constant along the longitudinal length of the body member. As can be appreciated, the body member can have a varying first diameter along at least a portion of the longitudinal length of the body member. The body member also has a second expanded diameter, not shown. The second diameter typically varies in size; however, the second diameter can be non-variable in size. The stent can be expanded in a variety of ways such as by a balloon. A balloon expandable stent is typically pre-mounted or crimped onto an angioplasty balloon catheter. A balloon catheter is then positioned into the patient via a guide wire. Once the stent is properly positioned, the balloon catheter is inflated to the appropriate pressure for stent expansion. After the stent has been expanded, the balloon catheter is deflated and withdrawn, leaving the stent deployed at the treatment area. One or more surfaces of the stent can be treated so as to have generally smooth surfaces; however, this is not required. Generally, one or more ends of the stent are treated by filing, buffing, polishing, grinding, coating, and/or the like to remove or reduce the number of rough and/or sharp surfaces; however, this is not required. The smooth surfaces of the ends reduce potential damage to surrounding tissue as the stent is positioned in and/or expanded in a body passageway.

The stent as illustrated in FIG. 1 is typically designed to be inserted into a diseased area in a body passageway and to expand the diseased area to enable better or proper fluid flow through the body passageway; however, the stent can be used for other or additional reasons. In one specific non-limiting example, the stent can be used to open an obstructed blood vessel. The stent can include and/or be used with one or more chemical agents used to inhibit thrombosis, in-stent restenosis, vascular narrowing and/or restenosis after the stent has been inserted into the blood vessel; however, this is not required. The one or more chemical agents, when used, can also or alternatively be used to remove and/or dissolve lipids, fibroblast, fibrin, etc. from the blood vessel so as to at least partially clean the blood vessel of such substances in the region of the stent and/or down stream of the stent. As can be appreciated, the one or more chemical agents, when used, can have additional or other functions.

The stent can be formed of a variety of materials (e.g., metal, polymer, etc.). The particular configuration of the stent illustrated in FIG. 1 can be used with materials having higher strength and a lower ductility than conventional materials such as stainless steel or cobalt alloys; however, this is not required. One non-limiting example of a metal alloy having a lower ductility than stainless steel or cobalt alloys is an alloy of Mo and Re. The stent is typically formed of a uniform material throughout the length of the stent; however, this is not required.

Referring again to FIG. 1, the stent is an expandable stent that can be used to at least partially expand occluded segments of a body passageway; however, the stent can have other or additional uses. For example, the expandable stent can be used as, but not limited to, 1) a supportive stent placement within a blocked vasculature opened by transluminal recanalization, which are likely to collapse in the absence of an internal support; 2) forming a catheter passage through mediastinal and/or other veins occluded by inoperable cancers; 3) reinforcing a catheter creating intrahepatic communication between portal and/or hepatic veins in patients suffering from portal hypertension; 4) a supportive stent placement of narrowing of the esophagus, the intestine, the ureter and/or the urethra; and/or 5) a supportive stent reinforcement of reopened and previously obstructed bile ducts. Accordingly, use of the term “stent” encompasses the foregoing or other usages within various types of body passageways, and also encompasses use for expanding a body passageway. The stent can be implanted or applied in a body passageway by techniques such as, but not limited to, balloon delivery, sheath catheter delivery, etc.

The stent can be formed by one or more processes such as, but not limited to, laser cutting, etching, EDM, wire welding, crimping, annealing, drawing, pilgering, electroplating, electropolishing, chemical polishing, cleaning, pickling, ion beam deposition or implantation, sputter coating, vacuum deposition, microelectromechanical manufacturing techniques etc. Once the stent is formed and/or cut, the stent can be further processed; however, this is not required. The one or more processes can include, but are not limited to, 1) electropolishing the stent, 2) treating one or more surfaces of the stent to create generally smooth surfaces (e.g., filing, buffing, polishing, grinding, coating, etc.), 3) at least partially coating the stent with one or more chemical agents, 4) at least partially coating the stent with one or more polymers, 5) forming one or more surface structures and/or micro-structures on one or more portions of the stent, and/or 6) inserting one or more markers on one or more portions of the stent.

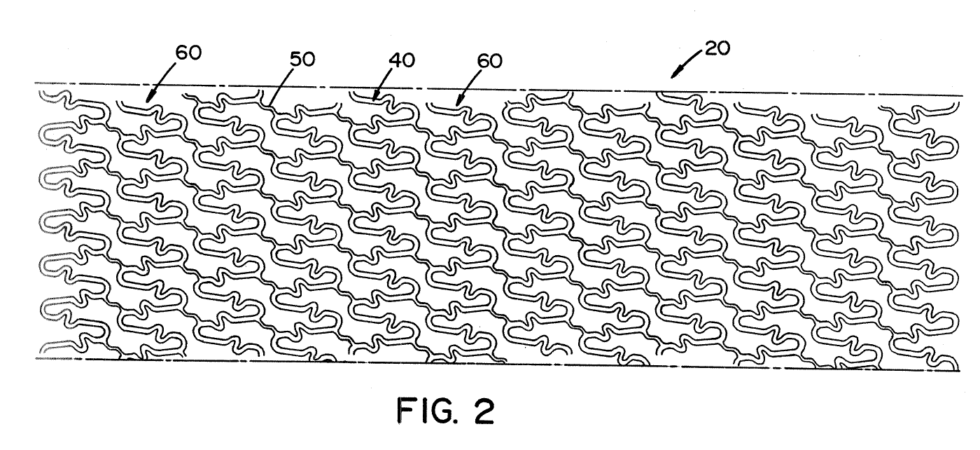

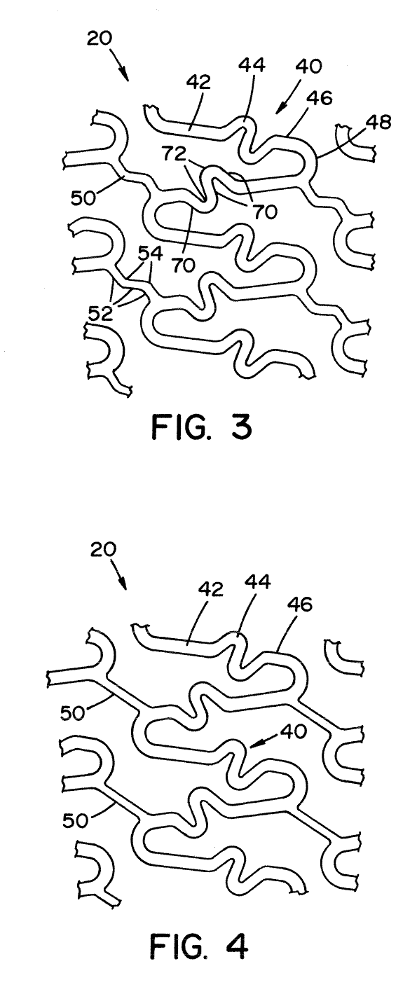

Referring now to FIGS. 2 and 3, stent 20 is formed by a plurality of rings 60 of struts 40. The rings of struts are connected together by a plurality of connectors 50. Each ring 60 of struts is formed by a plurality of struts. As best shown in FIG. 3, most, if not all, of the struts each include a generally first straight segment 42, undulating segment 44, a second generally straight segment 46, and an elbow or hinge segment 48. The undulating segment 44 is formed of a three generally straight portions 70 and two curved portions 72. As can be appreciated, one or more of the generally straight segments can be curved. As can also be appreciated the strut can eliminate the use of generally straight segments or generally curved segments, or one or more struts can have more than two generally straight segments or generally curved segments. As can further be appreciated, each strut can include more than one undulating segments. As can even further be appreciated, one or more of the undulating segments of the strut can include more than two curved portions. As can still further be appreciated, one or more of undulating segments 44 can be formed of more than three generally straight portions 70. As can yet further be appreciated, one or more of the generally straight portions 70 on one or more of undulating segments 44 can be non-straight. The connector 50 is shown to be connected to the elbow segment of two different struts. The connector is also shown to be an undulating component formed of a three generally straight portions 52 and two curved portions 54. As can be appreciated, one or more connectors can include more than one undulating segments. As can also be appreciated, one or more of the undulating segments of the connector can includes more than two curved portions. As can further be appreciated, one or more of undulating segments of the connector can be formed of more than three generally straight portions. As can still further be appreciated, one or more of the generally straight portions on the connector can be non-straight. As can also be appreciated, the connector can include one or more generally straight or generally curved segments in combination with one or more undulating segments. The width of connectors 50 is typically less than the width of strut 40 (e.g, maximum width of connector less than maximum width of strut, width of curved portions of connector less than width of generally straight segments of strut, etc.); however, this is not required. The width of connector 50 can be uniform, or vary in certain regions of the connector (e.g., width of curved portions narrower than width of generally straight portions, etc.). This novel design of the stent allows for deformation of the stent to occur during the expansion of the stent at the elbow sections of the struts as well as along the length of the struts, thus reducing the maximum stress at the elbow sections, thereby distributing the stresses beyond the elbow sections. The distribution of stresses is in part achieved by use of the undulating section 44 in the strut. The undulating section of the strut makes the stent more flexible. The length of connectors 50 can also be shortened, if desired, without reducing stent flexibility. The reduction of the length of the connectors can be used to accommodate more number of rings of struts per length of the stent, thereby reducing the open spaces in the body of the stent and/or increasing the radial strength of the stent. The use of the undulating sections in the struts also can reduce foreshortening of the stent after expansion.

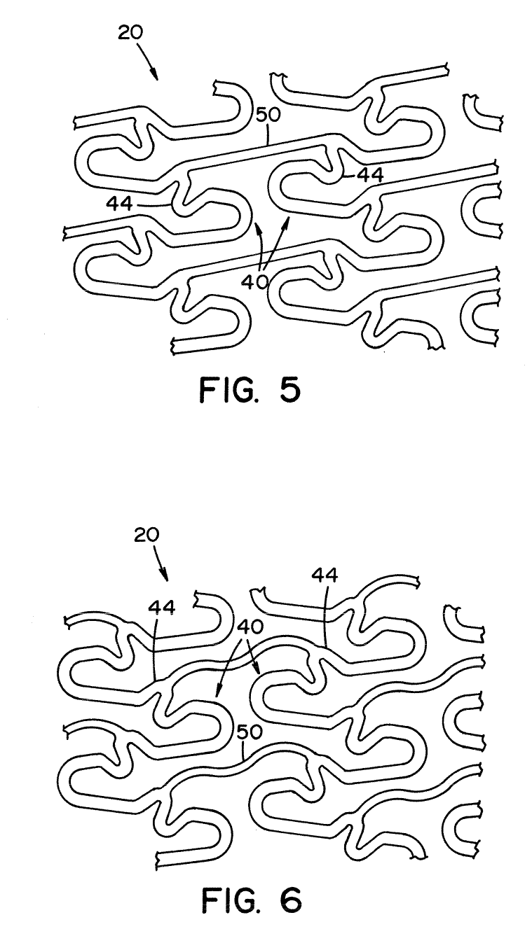

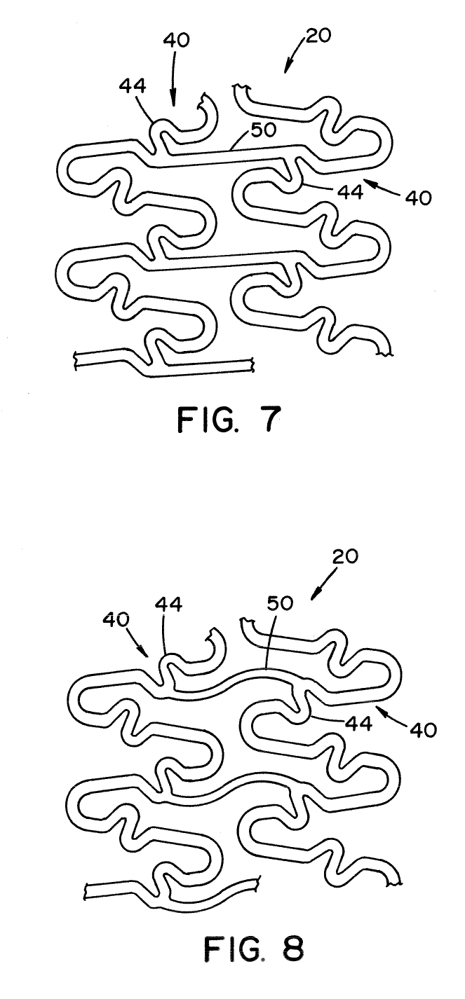

Referring now to FIGS. 4-9 and 11-14, various modifications of the inventions are illustrated. FIG. 4 illustrates connector 50 as a generally a straight structure. FIGS. 5 and 7 illustrate connector 50 as a generally straight structure that is connected to the undulating segments 44 of two struts 40. FIGS. 6 and 8 illustrate connector 50 as an undulating structure that is connected to the undulating segments 44 of two struts 40. As can be appreciated, one end of the connector could be connected to an undulating segment of one strut and the other end of the connector could be connected to a straight segment or elbow segment of another strut. In this configuration, the connector can be generally straight, undulating, etc. As can also be appreciated, one end of the connector could be connected to a generally straight segment of one strut and the other end of the connector could be connected to a straight segment, undulating segment or elbow segment of another strut. In this configuration, the connector can be generally straight, undulating, etc.

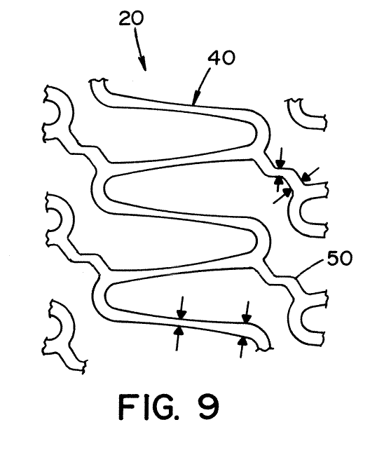

Referring now to FIG. 9, struts 40 are illustrated as having a varying width along a portion of the strut. FIG. 9 also illustrates that connector 50 as a varying width along a portion of the connector. The varying of the width of the connector and/or the strut can occur at one or more locations on the connector and/or the strut. As can be appreciated, the width of one or more connectors on the stent can be varied while the width of one or more struts remains constant. As also can be appreciated, the width of one or more struts on the stent can be varied while the width of one or more connectors remains constant. As can be further appreciated, one or more struts in FIG. 9 can include an undulating segment and/or one or more connectors can be generally straight.

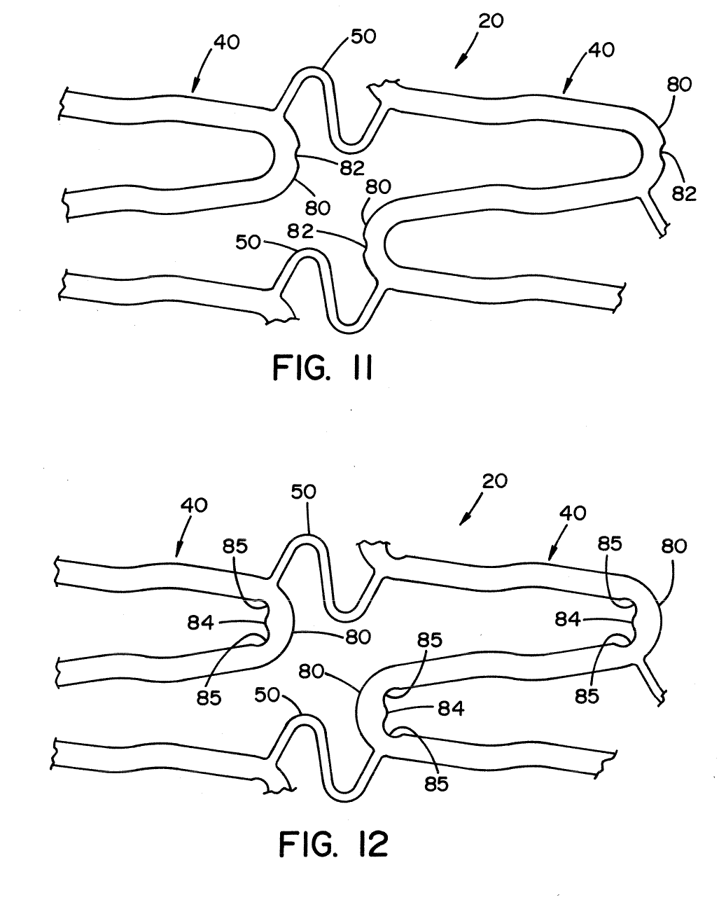

Referring now to FIG. 11, the curved apex 80 of struts 40 include at least one divot 82 on the outside edge or top surface of the strut. The divot is designed to facilitate in the bending of the apex during the expansion and/or contraction of the stent, and/or to redistribute stress on the apex when the apex is bent during the expansion and/or contraction of the stent. As can be appreciated, one or more or all of the divots 82 can be positioned on the back side or inner edge of the strut; however, this is not required. The connectors 50 are illustrated has have an undulating portion. The strut 40 is also illustrated as including an undulating portion.

Referring now to FIG. 12, the curved apex 80 of struts 40 include at least one dimple 84 on the back side or inner edge of the strut. The dimple is designed to redistribute stress on the apex when the apex is bent during the expansion and/or contraction of the stent. As can be appreciated, one or more or all of the dimples 84 can be positioned on the outside edge or top surface of the strut; however, this is not required. As illustrated in FIG. 12, the regions on both sides of the dimple can be narrow regions 85 that are narrower that the average thickness of the strut. These narrow regions are not required. The narrow regions when used can be on one or both sides of the dimple. The connectors 50 are illustrated has have an undulating portion. The strut 40 is also illustrated as including an undulating portion.

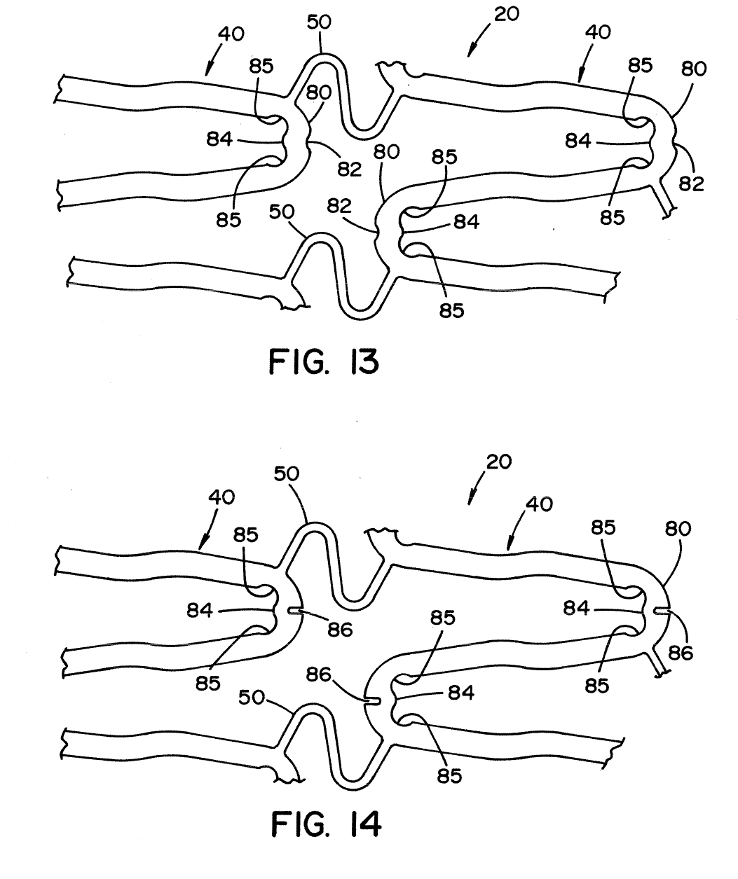

Referring now to FIG. 13, the curved apex 80 of struts 40 include at least one divot 82 on the outside edge or top surface of the strut and at least one dimple 84 on the back side or inner edge of the strut. The divot is designed to facilitate in the bending of the apex during the expansion and/or contraction of the stent and/or to redistribute stress on the apex when the apex is bent during the expansion and/or contraction of the stent. The dimple is designed to redistribute stress on the apex when the apex is bent during the expansion and/or contraction of the stent. The combined use of dimples and divots can improve the ease of bending the apex of the strut during the expansion and/or contraction of the stent, and/or improve the redistribution of stresses on the apex when the apex is bent during the expansion and/or contraction of the stent. When one dimple and divot are included on the apex of one or more struts, the dimple and divot are generally positioned on opposites sides of the apex from one another and exactly opposite from one another on the apex; however, this is not required. As can be appreciated, the number of divots and dimples on an apex of the strut can be the same or different. When different numbers of divots and dimples are included on an apex, generally none or the dimples and divots are positioned exactly opposite from one another on apex from one another; however, this is not required. As can be appreciated, one or more or all of the divots 82 can be positioned on the back side or inner edge of the strut; however, this is not required. As can also be appreciated, one or more or all of the dimples 84 can be positioned on the outside edge or top surface of the strut; however, this is not required. As illustrated in FIG. 13, the regions on both sides of the dimple can be narrow regions 85 that are narrower that the average thickness of the strut. These narrow regions are not required. The narrow regions when used can be on one or both sides of the dimple. The connectors 50 are illustrated has have an undulating portion. The strut 40 is also illustrated as including an undulating portion.

Referring now to FIG. 14, the curved apex 80 of struts 40 include at least one slot 86 on the outside edge or top surface of the strut and at least one dimple 84 on the back side or inner edge of the strut. The slot is designed to facilitate in the bending of the apex during the expansion and/or contraction of the stent and/or to redistribute stress on the apex when the apex is bent during the expansion and/or contraction of the stent. Due to the design of the slot, the slot facilitates in the bending of the apex throughout the contraction of the stent. During the expansion of the stent, the slot facilitates in the bending of the apex partially through the expansion of the apex until the sides of the slot engage one another and thereby facilitate less in the further expansion of the apex. The dimple is designed to redistribute stress on the apex when the apex is bent during the expansion and/or contraction of the stent. The combined use of slots and divots can improve the ease of bending the apex of the strut during the expansion and/or contraction of the stent, and/or improve the redistribution of stresses on the apex when the apex is bent during the expansion and/or contraction of the stent. When one dimple and slot are included on the apex of one or more struts, the dimple and slot are generally positioned on opposites sides of the apex from one another and exactly opposite from one another on the apex; however, this is not required. As can be appreciated, the number of slots and dimples on an apex of the strut can be the same or different. When different numbers of slots and dimples are included on an apex, generally none or the dimples and slots are positioned exactly opposite from one another on apex from one another; however, this is not required. As can be appreciated, one or more or all of the slots 86 can be positioned on the back side or inner edge of the strut; however, this is not required. As can also be appreciated, one or more or all of the dimples 84 can be positioned on the outside edge or top surface of the strut; however, this is not required. As illustrated in FIG. 14, the regions on both sides of the dimple can be narrow regions 85 that are narrower that the average thickness of the strut. These narrow regions are not required. The narrow regions when used can be on one or both sides of the dimple. The connectors 50 are illustrated has have an undulating portion. The strut 40 is also illustrated as including an undulating portion.

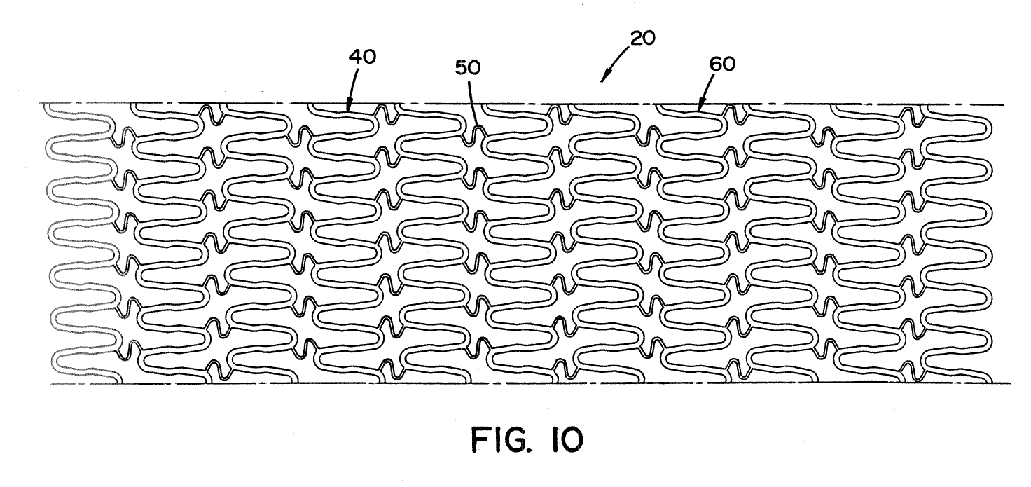

Referring to FIG. 10, another stent 20 is illustrated. The stent is formed by a plurality of rings 60 of struts 40. The rings of struts are connected together by a plurality of connectors 50. Each ring 60 of struts is formed by a plurality of struts. Most, if not all, of the struts each include a generally first straight segment 42, undulating segment 44, a second generally straight segment 46, and an elbow or hinge segment 48. The width of connectors 50 is typically less than the width of strut 40 (e.g, maximum width of connector less than maximum width of strut, width of curved portions of connector less than width of generally straight segments of strut, etc.); however, this is not required. The width of connector 50 can be uniform, or vary in certain regions of the connector (e.g., width of curved portions narrower than width of generally straight portions, etc.). This novel design of the stent allows for deformation of the stent to occur during the expansion of the stent at the elbow sections of the struts as well as along the length of the struts, thus reducing the maximum stress at the elbow sections, thereby distributing the stresses beyond the elbow sections. The distribution of stresses is in part achieved by use of the undulating section 44 in the strut. The undulating section of the strut makes the stent more flexible. The length of connectors 50 can also be shortened, if desired, without reducing stent flexibility. The reduction of the length of the connectors can be used to accommodate more number of rings of struts per length of the stent, thereby reducing the open spaces in the body of the stent and/or increasing the radial strength of the stent. The use of the undulating sections in the struts also can reduce foreshortening of the stent after expansion.

The stent illustrated in FIGS. 2 and 10 can have a variety of lengths. Generally the length is dependant on the location of placement of the stent in a body passageway. The maximum width ratio of the struts to the connectors is at least about 1.2:1, typically at least about 1.5:1, more typically about 1.5-4:1, and even more typically about 1.6-2.5:1. For example, a stent having a length of about 0.6-0.7 inches, can have an average strut width of about 0.0022-0.0045 inches and an average connector width of about 0.0012-0.003 inches. As stated above, the width of one or more struts and/or one or more connections of the stent can vary along the length of the strut and/or connector. The width ratio of such varying thickness between the maximum and minimum width is about 1.01-4:1, typically about 1.02-2.5:1, and more typically about 1.05-1.8:1. As can be appreciated, the change in width along the length of the strut and/or connector can a) gradually increase or decrease along the length of the strut and/or connector, b) abruptly increase or decrease along the length of the strut and/or connector, c) increase and then decrease one or more times along the length of the strut and/or connector, d) decrease and then increase one or more times along the length of the strut and/or connector, etc.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

1-67. (canceled)

68. An expandable medical device for use within in a body passageway comprising at least two struts and a connector securing together said two struts, said at least two struts spaced apart from one another, a plurality of said struts including an elbow section and an undulating section.

69. The expandable medical device as defined in claim 68, wherein said undulating section is connected to said elbow section.

70. The expandable medical device as defined in claim 68, wherein said undulating section is spaced from said elbow section.

71. The expandable medical device as defined in claim 68, wherein said elbow section has a radius of curvature of about 100-350°.

72. The expandable medical device as defined in claim 68, wherein a plurality of said struts have a non-uniform width.

73. The expandable medical device as defined in claim 72, wherein said elbow on a plurality of said struts has a non-uniform width.

74. The expandable medical device as defined in claim 72, wherein said undulating section on a plurality of said struts has a non-uniform width.

75. The expandable medical device as defined in claim 73, wherein said undulating section on a plurality of said struts has a non-uniform width.

76. The expandable medical device as defined in claim 68, wherein a plurality of said connectors has a non-uniform width.

77. The expandable medical device as defined in claim 75, wherein a plurality of said connectors has a non-uniform width.

78. The expandable medical device as defined in claim 68, wherein a plurality of said connectors includes an undulating section.

79. The expandable medical device as defined in claim 68, wherein at least one end of said connector is connected to said elbow section of one of said struts.

80. The expandable medical device as defined in claim 68, wherein at least one end of said connector is connected to said undulating section of one of said struts.

81. The expandable medical device as defined in claim 68, wherein said medical device is a stent.

82. The expandable medical device as defined in claim 68, wherein an apex on a plurality of said elbow sections includes at least one dimple on one side of said elbow sections.

83. The expandable medical device as defined in claim 68, wherein an apex on a plurality of said elbow sections includes at least one at least one a divot, slot, and combinations thereof.

84. The expandable medical device as defined in claim 68, wherein an apex on a plurality of said elbow sections includes at least one dimple and at least one a divot, slot, and combinations thereof on a same side of elbow sections.

85. The expandable medical device as defined in claim 68, wherein an apex on a plurality of said elbow sections includes at least one dimple on one side of said elbow sections and at least one a divot, slot, and combinations thereof on an opposite side of elbow sections.

86. The expandable medical device as defined in claim 84, wherein an apex on a plurality of said elbow sections includes at least one dimple on one side of said elbow sections and at least one a divot, slot, and combinations thereof on an opposite side of elbow sections.

87. The expandable medical device as defined in claim 82, wherein the apex includes at least one narrow region on at least one side of said dimple on a plurality of said elbows.

88. The expandable medical device as defined in claim 68, including one or more chemical agents.

89. The expandable medical device as defined in claim 88, including one or more polymers to at least partially control a release rate of said chemical agents from a plurality of said struts, a plurality of said connector, and combinations thereof.

90. The expandable medical device as defined in claim 68, wherein at least one component of said medical device includes molybdenum, rhenium, and mixtures thereof.
